(19) United States
(12) Patent Application Publication
(10) Pub. No.: US 2019/0365673 A1
(43) Pub. Date: Dec. 5, 2019

(54) Immunomodulatory Compounds

(71) Applicants: Lankenau Institute for Medical Research, Wynnewood, PA (US); University of Central Florida Research Foundation, Inc., Orlando, FL (US)

(72) Inventors: Susan Gilmour, Wynnewood, PA (US); Otto Phanstiel, Oviedo, FL (US)

(21) Appl. No.: 16/468,043

(22) PCT Filed: Dec. 8, 2017
(86) PCT No.: PCT/US2017/065305

§ 371 Date: Jun. 10, 2019

Related U.S. Application Data
(60) Provisional application No. 62/431,663, filed on Dec. 8, 2016.

(51) Int. Cl. A61K 31/135; A61K 31/197; A61P 35/00

(52) U.S. Cl. CPC A61K 31/135; A61P 35/00; A61K 31/197

(57) ABSTRACT

Compositions and methods for modulating the immune response in a subject are disclosed.

The present application claims priority to U.S. Provisional Application No. 62/431,663, filed Dec. 8, 2016. The entire disclosure of the foregoing applications is incorporated by reference herein.

This invention was made with government support under Grant No. RO1 CA70739 awarded by the National Institutes of Health. The government has certain rights in the invention.

### FIELD OF THE INVENTION

This invention relates generally to the field of immunomodulation. Specifically, the invention provides compositions and methods for modulating the immune response in a subject.

### BACKGROUND OF THE INVENTION

Cancer cells develop many diverse and complex mechanisms to evade the effects of chemotherapeutics, particularly drugs that target a specific signaling pathway (Rebucci, et al. (2013) Biochem. Pharmacol., 85:1219-26). This chemoresistance remains a significant obstacle to successful cancer therapy. Given the heterogeneity and plastic nature of most tumors, a better approach may be to target tumor modifiers that support multiple components of the tumor including both tumor epithelial cells as well as stromal and infiltrating immune cells in the tumor microenvironment (Dobbelstein et al. (2014) Nat. Rev. Drug Discov., 13:179-96; Zhao, J. (2016) Pharmacol. Ther., 160:145-58; Colotta et al. (2009) Carcinogenesis 30:1073-81). Compared to normal cells, tumor cells have been shown to contain elevated levels of polyamines (putrescine, spermidine, and spermine) (Pegg, A. E. (1988) Cancer Res., 48:759-74; Tabor et al. (1984) Ann. Rev. Biochem., 53:749-90; Pegg, A. E. (1986) Biochem. J., 234:249-62; Gerner et al. (2004) Nat. Rev. Cancer 4:781-92). Polyamines are amino acid-derived polycations that have been implicated in a wide array of biological processes, and they are essential for cellular proliferation, differentiation, and cell death (Gerner, et al. (2004) Nat. Rev. Cancer 4:781-92; Cufi et al. (2011) Cell Cycle 10:3871-85; Mirzoeva et al. (2011) J. Mol. Med., 89:877-89; Morselli et al. (2011) J. Cell. Biol., 192:615-29; Morselli et al. (2011) Antioxid. Redox Signal. 14:2251-69; Wallace et al. (2003) Biochem. J., 376:1-14; Thomas et al. (2003) J. Cell Mol. Med., 7:113-26; Wei et al. (2007) Mol. Carcinog., 46:611-7; Pegg, A. E. (2009) IUBMB Life 61:880-94). Intracellular polyamine levels are maintained via tightly-regulated biosynthetic, catabolic, and uptake and export pathways (Wallace et al. (2003) Biochem. J., 376:1-14). Oncogenes such as MYC and RAS both upregulate polyamine biosynthesis (Bello-Fernandez et al. (1993) Proc. Natl. Acad. Sci., 90:7804-8; Forshell et al. (2010) Cancer Prev. Res., 3:140-7; Origanti et al. (2007) Cancer Res., 67:4834-42) and increase cellular uptake of polyamines by inducing the polyamine transport system (PTS) (Bachrach et al. (1981) Cancer Res., 41:1205-8; Chang et al. (1988) Biochem. Biophys. Res. Commun., 157:264-70). In order to meet their huge metabolic needs, most tumors have a greatly increased need for polyamines compared to normal cells and, consequently, polyamines are potent modifiers of tumor development (Seiler et al. (1996) Int. J. Biochem. Cell. Biol., 28:843-61).

Targeting polyamine metabolism has long been an attractive approach to cancer chemotherapy. Ornithine decarboxylase (ODC), the rate-limiting enzyme in polyamine biosynthesis, is elevated in tumors and is an early marker and promoter of tumorigenesis (Gerner et al. (2004) Nat. Rev. Cancer 4:781-92; O'Brien et al. (1997) Cancer Res., 57:2630-7; Lan et al. (2005) J. Invest. Dermatol., 124:602-14; Lan et al. (2000) Cancer Res., 60:5696-703; Smith et al. (1998) Carcinogenesis 19:1409-15). Although α-difluoromethylornithine (DFMO), an irreversible inhibitor of ODC activity, showed promise as a chemotherapeutic agent in vitro, it has had only moderate success in treating cancer patients (Gerner et al. (2004) Nat. Rev. Cancer 4:781-92). Subsequent studies discovered that its effect on inhibiting polyamine biosynthesis was abrogated in vivo with a compensatory increased activity of the PTS in tumor cells with resulting increased uptake of polyamines derived from the diet and gut flora into the tumor cells (Seiler et al. (1996) Int. J. Biochem. Cell. Biol., 28:843-61; Thomas et al. (2001) Cell Mol. Life Sci., 58:244-58). Thus, new methods of targeting polyamine metabolism are needed.

### SUMMARY OF THE INVENTION

In accordance with one aspect of the instant invention, compositions and methods for inhibiting, treating, and/or preventing cancer in a subject are provided. Methods for improving or enhancing the therapeutic efficacy (e.g., anti-cancer properties) of an anti-cancer therapy are also provided. In a particular embodiment, the methods of the instant invention comprise administering an aryl based polyamine transport system inhibitor to the subject. In a particular embodiment, the polyamine transport system inhibitor is administered with and an anti-cancer therapy (consecutively and/or concurrently). In a particular embodiment, the method further comprises administering an ornithine decarboxylase inhibitor (e.g., α-difluoromethyl-ornithine (DFMO)). In a particular embodiment, the cancer is metastatic. In a particular embodiment, the cancer is melanoma. In a particular embodiment, the cancer comprises CXCR4 positive cells. In a particular embodiment, the cancer is resistant to the anti-cancer therapy (in the absence of the polyamine transport system inhibitor and/or ornithine decarboxylase inhibitor). Examples of anti-cancer therapies include, without limitation: chemotherapy, radiation therapy, surgery, and/or immunotherapy.

In accordance with another aspect of the instant invention, compositions and methods for modulating the immune system (e.g., enhancing or increasing the immune response to challenge) in a subject are provided. In a particular embodiment, the methods of the instant invention comprise administering an aryl based polyamine transport system inhibitor to the subject. In a particular embodiment, the method further comprises administering an ornithine decarboxylase inhibitor (e.g., α-difluoromethyl-ornithine (DFMO)).

### BRIEF DESCRIPTION OF THE FIGURES

FIGS. 1A-1E show that B16F10-sTAC tumor growth inhibition with DFMO and Trimer PTI. FIG. 1A: Mice were subcutaneously injected with 5×10⁵ B16F10-sTAC melanoma cells. When tumors were 50-100 mm³ in size, treatment was initiated with either saline, 0.25% DFMO (w/v) in the drinking water, Trimer PTI (i.p., 3 mg/kg, once a day) or both DFMO and Trimer PTI. Graph shows B16F10-sTAC tumor growth under different treatments (mean tumor volume±SEM). FIG. 1B: Upon sacrifice, tumors were excised and weighed (mean±SEM). FIG. 1C: Spleen weight was determined upon sacrifice (mean±SEM). FIG. 1D: Polyamine levels were determined in tumors by HPLC and normalized to DNA levels in the tissue extracts (nmol/mg DNA). FIG. 1E: Tumor and non-tumor bearing skin tissues were excised from PBT-treated mice, flash frozen, finely pulverized in liquid nitrogen with mortar and pestle, homogenized in a solution of 33% water, 66% methanol and 1% acetic acid, and then centrifuged at 5000 rpm for 10 minutes at 25° C. Trimer PTI levels were determined in tumor and skin supernatants by mass spectrometry and normalized to tissue protein concentration (nmol/mg protein). n=5-10 mice per group; *=p≤0.05 and #=p≤0.01 compared to vehicle-treated mice.

FIGS. 2A-2D show that DFMO and Trimer PTI co-treatment increases cytotoxic T-cell activity and promotes macrophage infiltration of the tumor. FIG. 2A: The frequency of IFN-γ producing T-cells was measured by the ELISpot assay as IFN-γ spot forming units (SFU) per million spleen cells. FIG. 2B: The number of F4/80⁺ cells per million B16F10-sTAC melanoma cells from either vehicle treated mice or mice cotreated with DFMO and Trimer PTI. Representative images of B16F10-sTAC tumor sections from mice treated with vehicle (FIG. 2C) or DFMO and Trimer PTI (FIG. 2D) and stained for F4/80⁺ macrophages. n=5-10 mice per group; *=p≤0.05 and #=p≤0.01 compared to vehicle treated mice.

FIG. 3 shows that DFMO and Trimer PTI co-treatment increases levels of pro-inflammatory cytokines in B16F10-sTAC tumors. Upon sacrifice, tumors were excised, flash frozen in liquid nitrogen and then homogenized to produce tumor lysates which were assayed for levels of TNF-α, IL-10, IFN-γ, IL-6, MCP-1 and VEGF by Cytokine Bead Array or ELISA. n=5-10 mice per group; *=p≤0.05 and #=p≤0.01 compared to indicated group.

FIGS. 4A-4C show that depletion of CD4⁺ and CD8⁺ T-cells reverses PBT inhibition of tumor growth. FIG. 4A: Mice were subcutaneously injected with 5×10⁵ CT26.CL25 colon carcinoma cells. When tumors were 50-100 mm³ in size, treatment was initiated with either saline or 0.25% DFMO (w/v) in the drinking water plus Trimer PTI (i.p. 3 mg/kg, once a day). Mice in the anti-CD4/CD8 groups were i.p. injected with 75 μg of anti-CD4 and anti-CD8 antibodies every three days starting 3 days prior to the initiation of treatment with a total of four doses. Graph shows CT26.CL25 tumor growth under different treatments (mean tumor volume±SEM). Upon sacrifice, tumors (FIG. 4B) and spleens (FIG. 4C) were excised and weighed (mean±SEM). n=5-10 mice per group; *=p≤0.05 and #=p≤0.01 compared to indicated group.

FIG. 5A shows that DFMO and Trimer PTI co-treatment reduces the immunosuppressive and pro-tumorigenic cells populations. Upon sacrifice, tumors were excised from CT26.CL25-tumor bearing mice and processed for analysis by flow cytometry. CD45⁺ tumor cells were analyzed for the percentage of the indicated cell subpopulations. n=5-10 mice per group; *=p≤0.05 and #=p≤0.01 compared to indicated group. FIG. 5B shows that DFMO and Trimer PTI co-treatment reduces the immunosuppressive and pro-tumorigenic cells populations in the spleen. Upon sacrifice, spleens were excised from CT26.CL25-tumor bearing mice and processed for analysis by flow cytometry. CD45⁺ tumor cells were analyzed for the percentage of the indicated cell types. n=5-10 mice per group. *=p≤0.05 and #=p≤0.01 compared to indicated group.

FIGS. 6A-6C show that co-treatment with DFMO and Trimer PTI increases the cytotoxic T-cell activity in the tumor. Upon sacrifice, tumors were excised from CT26.CL25-tumor bearing mice and infiltrating leukocytes were isolated by discontinuous Percoll gradients. CD8⁺ T-cells were analyzed for the percentage of IFN-γ+(FIGS. 6A and 6B) and granzyme B⁺ (FIGS. 6A and 6C) cells by flow cytometry. FIG. 6D: The frequency of IFN-γ producing T-cells was measured by the ELISpot assay as IFN-γ spot forming units (SFU) per 5×10⁵ tumor infiltrating leukocytes. n=5-10 mice per group; *=p≤0.05 and #=p≤0.01 compared to indicated group.

FIGS. 7A and 7B show that co-treatment with DFMO and Trimer PTI decreases the immunosuppressive activity of MDSCs. FIG. 7A: One week prior to sacrifice, MDSCs were isolated from the spleens of CT26.CL25 tumor-bearing mice that were treated either with vehicle or co-treatment with DFMO and Trimer PTI decreases. Isolated MDSCs (5.2×10⁶ per mouse) were then adoptively transferred into separate groups of recipient tumor-bearing mice in the PBT-treated group. FIG. 7B: Upon sacrifice, splenocytes from the recipient mice were used to measure the frequency of IFN-γ producing T-cells by ELISpot assay following challenge with tumor-expressing β-galactosidase peptide. n=5-10 mice per group; *=p≤0.05 and #=p≤0.01 compared to indicated group.

FIGS. 8A-8C show greater PTS activity and increased sensitivity to AP in BRAF^{V600E} human melanoma cells compared to BRAF^{WT} cells. FIG. 8A: BRAF^{V600E} WM983B melanoma cells and BRAF^{WT} WM3743 melanoma cells were cultured with and without 1 mM DFMO for 40 hours and then pulsed with 1 μM ³H-spermidine for 60 minutes at 37° C. Cells were washed with cold PBS containing 50 M spermidine, and cell lysates were assayed for CPM ³H-spermidine per mg protein by scintillation counting. FIG. 8B: WM983B and WM3743 melanoma cells were treated with increasing doses of AP. After 72 hours of culture, cell survival was determined via EZQuant™ Cell Quantifying assay. IC₅₀ values were calculated by GraphPad Prism 6. FIG. 8C: WM983B melanoma cells were treated with increasing doses of AP with or without 1 mM DFMO. After 72 hours of culture, cell survival was determined via EZQuant™ Cell Quantifying assay. 72 hour IC₅₀ values were calculated by GraphPad Prism 6. Values are the mean of 5-6 samples+SD. p≤0.05 was considered statistically significant.

FIGS. 9A-9C show that BRAF^{V600E} murine melanoma cells are more sensitive to AP than BRAF^{WT} melanoma cells. FIG. 9A: Murine BRAF^{V600E} YUMM1.7 melanoma cells and BRAF^{WT} B16F10 melanoma cells were treated with increasing doses of AP. After 72 hours of culture, cell survival was determined via EZQuant™ Cell Quantifying assay. IC₅₀ values were calculated by GraphPad Prism 6. FIG. 9B: Murine BRAF^{V600E} YUMM1.7 melanoma cells and BRAF^{WT} B16F10 melanoma cells were treated with increasing doses of AP±1 mM DFMO or with PLX4720. After 72 hours of culture, cell survival was determined via EZQuant™ Cell Quantifying assay. IC₅₀ values were calculated by GraphPad Prism 6. FIG. 9C: YUMM1.7 and B16F10 melanoma cells and B16F10 cells retrovirally infected to express the mutant BRAF^{V600E} protein were cultured with and without 1 mM DFMO for 40 hours and then pulsed with 1 μM ³H-spermidine for 60 minutes at 37° C. Cells were washed with cold PBS containing 50 μM spermidine, and cell lysates were assayed for CPM ³H-spermidine per mg protein by scintillation counting.

FIG. 10 shows that increased resistance of spheroid melanoma cells to PLX4720 is overcome with AP co-treatment. BRAF^{V600E} mutant 1205Lu melanoma cells were seeded at 1×10⁴ cells in each well of the 24-well NCPs. When spheroids were formed on day 3, the 3D cultures of spheroids were treated with PLX4720 (25 μM) and/or AP (25 μM). 2D monolayer cultures of 1205Lu melanoma cells were also treated with PLX4720 (25 μM) and/or AP (25 μM). After drug treatment for 48 hours, the viability of spheroids and monolayer cultures was assayed using the CellTiter-Glo Luminescent Cell Viability Assay. The percent cell survival in each treatment group was calculated relative to cells treated with medium only under the same conditions. As controls, the growth of cells without drug treatment under each condition was normalized as 100% separately. The means are presented±SD.

FIG. 11 provides a graph of tumor volume in mice over time after treatment with PLX4720 alone or PLX4720 with delayed administration of AP and DFMO.

FIG. 12A shows tumor volume in C57Bl/6 mice injected with B16F10 melanoma cells and treated with either control or AP (Formula (II)). FIG. 12B shows percentage of F4/80⁺ CD206⁺ splenocytes from mice treated with vehicle, DFMO, AP, or AP+DFMO. FIG. 12C shows percentage of IFNγ-producing T cells after ionomycin and PMA stimulation of splenocytes from control or AP-treated mice.

FIG. 13 provides a graph showing stromal cell-derived factor 1 (SDF-1) stimulated migration of macrophage in the presence of AMD3100 or AP.

### DETAILED DESCRIPTION OF THE INVENTION

Polyamines are low molecular weight aliphatic amines and are essential metabolites for cell growth (Cullis et al. (1999) Chem. Biol., 6:717-729; Seiler et al. (1996) Int. J. Biochem., 28:843-861; Seiler et al. (1990) Int. J. Biochem., 22:211-218; Poulin et al. (2012) Amino Acids 42:711-723). Tumor cells have elevated polyamine levels and have active polyamine transport systems to import exogenous polyamines (Cullis et al. (1999) Chem. Biol., 6:717-729). The polyamine transport system (PTS) is an important target, as many cancer cells need to import polyamines in order to sustain their growth rate and for cell survival. To polyamine-starve a tumor, both polyamine biosynthesis as well as polyamine transport can be inhibited. Following the discovery that DFMO treatment upregulates the PTS in tumors, work has focused on finding drugs that can target the PTS.

To starve tumor cells of polyamines that are essential for their growth and survival, a new polyamine blockade therapy (PBT) that includes a combination of 1) a polyamine transport inhibitor (PTI) and, optionally, 2) DFMO is provided herein. This approach exploits the oncogene and DFMO-induced PTS activity in tumor cells by inhibiting the PTS with a novel Trimer PTI (Muth et al. (2013) J. Med. Chem., 56:5819-28; Muth et al. (2014) J. Med. Chem., 57:348-63). Both natural polyamines and polyamine-based drugs are imported into tumors via this specific polyamine uptake system. In contrast, normal cells are predicted to be significantly less sensitive to the Trimer PTI due to their low PTS activity (Phanstiel et al. (2007) Amino Acids 33:305-13). Herein, the anti-tumor efficacy of combination treatment with DFMO and the Trimer PTI in two animal tumor models is provided. It is shown that this polyamine-targeted therapy provides a dual attack on tumors by starving the tumors of the polyamine growth factors needed for their proliferation and survival and by activating an immune attack on these tumors.

The instant invention provides a new use for arylpolyamine drug-like compounds that target the polyamine transport system in cells for local and/or systemic immunomodulation. Polyamines are small amino acid-derived molecules found in all cells that are essential for all life processes. Levels of polyamines are dramatically increased in tumor cells compared to normal cells, due to both increased biosynthesis and increased uptake via the polyamine transport system (PTS). Three-armed polyamine derivatives of an aryl compound are effective polyamine transport system inhibitors (PTI) (Muth, et al. (2014) J. Med. Chem., 57(2):348-363). Further, 2-armed polyamine derivatives of an aryl compound are capable of gaining access inside cells via the PTS (Muth et al. (2013) J. Med. Chem., 56(14):5819-5828) and can be used as a “Trojan horse” drug to deliver a toxic entity into cells.

Without being bound by theory, both of these polyamine-derived drugs inhibit the uptake of polyamines by virtue of their polyamine tails that compete with normal polyamines for entry into the cell via the polyamine transport system (PTS) (Muth et al. (2013) J. Med. Chem., 56(14):5819-5828). To starve tumors of polyamines, a novel polyamine-based therapy is used herein that includes (1) an inhibitor of the rate-limiting enzyme in the polyamine biosynthesis pathway-ornithine decarboxylase (ODC) (e.g., α-difluoromethyl-ornithine (DFMO), an FDA-approved drug); and (2) a polyamine transport system inhibitor (PTI). This strategy results in starving the tumor cells of polyamines that are essential for their growth and survival.

As noted above, 2-armed polyamine derivatives of aryl compounds can act as a Trojan horse due to their ability to also gain entry into the cell via the PTS. Two-armed polyamine derivatives of aryl compounds may also have enhanced cytotoxic potency via their multiple electrostatic interactions with DNA (Dallavalle et al. (2006) J. Med. Chem., 49(17):5177-5186) and topoisomerase II (Wang et al. (2001) J. Med. Chem., 44(22):3682-3691). However, normal cells are significantly less sensitive to 2-armed polyamine derivatives of aryl compounds compared to tumor cells due to the very low PTS activity and polyamine uptake in normal cells (Muth et al. (2013) J. Med. Chem., 56(14):5819-5828).

Polyamine levels are elevated in tumors and are released by dying cancer cells found in hypoxic and necrotic areas of the tumor following chemotherapy or radiotherapy. In vivo evidence indicates that the anti-inflammatory effects of polyamines released into the tumor microenvironment contribute to the immunosuppressive milieu commonly found in most tumors. Using multiple murine tumor models, it has been shown that: i) elevated polyamine-mediated suppression of an antigen-specific immune response creates a more permissive microenvironment for tumor growth; ii) inhibition of tumor growth by polyamine depletion in tumors via co-treatment with DFMO and a polyamine transport inhibitor is T-cell-dependent; and iii) treatment with DFMO and a polyamine transport inhibitor before surgical removal of the primary tumor leads to protective immunity to tumor re-challenge. Moreover, polyamines have been shown to inhibit induction of proinflammatory cytokines in monocytes, protect against lethal sepsis, and suppress immune cell activity (Zhang et al. (2000) Crit. Care Med. 28(4 Suppl):N60-66; Zhu et al. (2009) Mol. Med., 15(7-8):275-282; Chamaillard et al. (1993) Anticancer Res., 13(4):1027-1033; Chamaillard et al. (1997) Br. J. Cancer 76(3):365-370). Moreover, polyamines have been shown to suppress adaptive immune responses. Using transgenic mice in which ornithine decarboxylase activity is specifically targeted to the epidermis, elevated polyamine levels were shown to potently suppress a hapten-induced contact allergic response, which is T-cell mediated (Keough et al. (2011) J. Invest. Dermatol., 131(1):158-166). Thus, the role of polyamines as local anti-inflammatory effector molecules at sites of infection or wounds is usurped by tumors to provide a survival mechanism to evade the immune response.

CXCR4 plays an important role in the recruitment and polarization of tumor associated macrophages (TAM) that can promote therapy-resistance and tumor progression (Lee et al. (2006) Mol. Cancer Ther., 5(10): 2592-2599). Data provided herein shows that polyamine transport system inhibitors of the instant invention not only inhibit uptake of polyamines but also inhibit CXCR4/stromal cell-derived factor-1 (SDF-1) signaling, which is a critical regulator of tumor-stromal interactions driving metastasis. Polyamine transport system inhibitors of the instant invention may also target M2 macrophages that highly express CXCR4 and have been found to contribute to immuno/chemotherapeutic resistance and to indirectly facilitate tumor progression and metastasis (Hughes et al. (2015) Cancer Res., 75(17):3479-91; Beider et al. (2014) Oncotarget 5(22): 11283-11296). Because anti-cancer therapies cause tumor necrosis, vascular damage, and hypoxia, all of these can upregulate myeloid cell chemoattractants including SDF-1 in the tumor microenvironment and promote polarization of TAMs toward a pro-tumorigenic M2 phenotype (Hughes et al. (2015) Cancer Res., 75(17):3479-91; De Palma et al. (2013) Cancer Cell 23(3):277-286; Russell et al. (2013) Front. Physiol., 4:157). Therefore, the polyamine transport system inhibitors of the instant invention have the dual activity as not only a polyamine transport inhibitor but also an inhibitor of CXCR4 signaling that is putatively responsible for metastasis (Kim et al. (2010) Cancer Res., 70(24): 10411-10421), resistance to immunotherapy (Lee et al. (2006) Mol. Cancer Ther., 5(10): 2592-2599), and the recruitment and M2 polarization of TAMs that can promote therapy-resistance and tumor progression (Hughes et al. (2015) Cancer Res., 75(17):3479-91; Beider et al. (2014) Oncotarget 5(22):11283-11296).

In accordance with the instant invention, methods of stimulating the immune system of a subject (e.g., stimulating, increasing, and/or enhancing an immune response and/or reaction in the subject) are provided. In a particular embodiment, the method reduces suppression of the immune system (e.g., immunosuppression) in a subject. The suppression of the immune system of a subject may be due to a disease state such as, without limitation, cancer or microbial infection (e.g., bacterial infection, viral infection, or fungal infection).

In a particular embodiment, the method comprises administering a polyamine transport system inhibitor to the subject, wherein the polyamine transport system inhibitor is a polyamine aryl compound. In a particular embodiment, the polyamine transport system inhibitor has the structure:

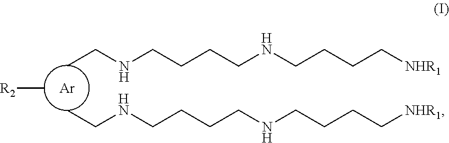

wherein each R₁ is independently H or methyl, wherein Ar is an aryl group, and wherein R₂ is H or

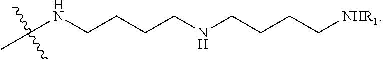

In a particular embodiment, each R₁ is methyl. In a particular embodiment, R₂ is H. The polyamine arms of the compound may be attached to chemically feasible position of the aryl group, particularly to an atom (e.g., carbon) of the aromatic ring(s). In a particular embodiment, when R₂ is H, the polyamine arms of the compound are attached to opposite sides of an aromatic ring. In a particular embodiment, the polyamine arms of the compound are attached to an aromatic ring such that they are equally spaced from each other (e.g., at position 1, 3, and 5 or 2, 4, and 6 of a six-membered ring, when there are three polyamine arms).

An aryl group, as used herein, refers to monocyclic, bicyclic, and tricyclic aromatic groups containing about 5 to about 18 carbons in the ring portion(s). Aryl groups may be optionally substituted through available carbon atoms (e.g., may include 1 to about 4 substituents). Exemplary substituents may include, but are not limited to, alkyl, halo, haloalkyl, alkoxyl, alkylthio, hydroxyl, methoxy, carboxyl, oxo, epoxy, alkyloxycarbonyl, alkylcarbonyloxy, amino, carbamoyl, urea, alkylurea, and thiol. Aryl groups may comprise a ring system that includes at least one sulfur, oxygen, or nitrogen heteroatom ring members (i.e., the aryl group may be a heteroaryl group). Examples of aryl groups include, without limitation, benzene, naphthalene, indole, pyridine, pyrrole, furan, thiopene, pyrazole, imidazole, oxazole, isoxaole, thiazole, isothiazole, triazole, oxadiazole, furazan, thiadiazole, pyridazine, pyrimidine, pyrazine, oxazine, dioxine, thiazine, triazine, pentalene, pyrrolizine, indolizine, benzofuran, benzothiopene, indazole, benzimidazole, benzthiazole, purine, quinoline, quinolizine, quinazoline, benzopyrazine, naphthyridine, phthalazine, pteridine, acenaphthylene, carbazole, anthracene, phenanthrene, acridine, phenazine, and phenanthroline. In a particular embodiment, the aryl group is selected from the group consisting of benzene, naphthalene, and anthracene.

In a particular embodiment, the polyamine transport system inhibitor has the structure:

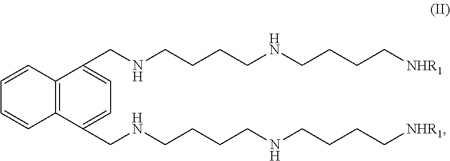

wherein each R₁ is independently H or methyl.

In a particular embodiment, the polyamine transport system inhibitor has the structure:

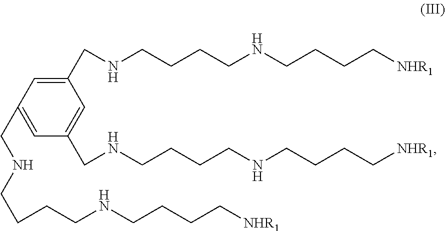

wherein each R₁ is independently H or methyl.

The methods of the instant invention may also further comprise administering an inhibitor of omithine decarboxylase. Ornithine decarboxylase catalyzes the rate-limiting conversion of ornithine to putrescine of the polyamine biosynthesis pathway. Notably, increased ornithine decarboxylase activity has been associated with increased tumor growth. By inhibiting polyamine biosynthesis (e.g., with an inhibitor of ornithine decarboxylase) and inhibiting polyamine transport (e.g., with an polyamine transport system inhibitor), the amount of polyamine available to a cell is greatly reduced. Ornithine decarboxylase inhibitors include, without limitation, α-difluoromethylomithine (DFMO; also known as eflornithine), N-(4′-Pyridoxyl)-ornithine(BOC)-OMe (POB; Wu et al. (2007) Mol. Cancer Ther., 6:1831), α-methyl ornithine, 1,4-diamino-2-butanone (DAB), and 1,3-diaminopropane. In a particular embodiment, the omithine decarboxylase inhibitor is DFMO.

In accordance with another aspect of the instant invention, methods for treating, inhibiting, and/or preventing cancer in a subject are provided. The instant invention also encompasses methods of improving the therapeutic efficacy of an anti-cancer therapy. These methods comprise administering a polyamine transport system inhibitor, as described above. In a particular embodiment, these methods comprise administering a polyamine transport system inhibitor (optionally with an ornithine decarboxylase inhibitor), as described above, in combination with an anti-cancer therapy. In other words, the polyamine transport system inhibitor (optionally with an ornithine decarboxylase inhibitor) is used as an adjunct therapy. For example, the polyamine transport system inhibitor (optionally with an omithine decarboxylase inhibitor) can be administered in combination with chemotherapy (e.g., the administration of at least one chemotherapeutic agent), radiation therapy, surgery to remove cancerous cells or a tumor (e.g., resection), and/or immunotherapy. The therapies may be administered consecutively (before or after) and/or at the same time (concurrently) as the polyamine transport system inhibitor (optionally with an ornithine decarboxylase inhibitor). Co-administered agents may be administered in the same composition or in separate compositions.

In a particular embodiment, the cancer being treated is resistant to the anti-cancer therapy being administered. However, the co-administration of a polyamine transport system inhibitor (optionally with an ornithine decarboxylase inhibitor) renders the resistant cancer susceptible to the treatment of the instant invention. In a particular embodiment, the cancer being treated is metastatic. In a particular embodiment, the cancer being treated comprises CXCR4 positive cells (e.g., CXCR4 positive cancer stem cells).

In a particular embodiment, the cancer that may be treated using the compositions and methods of the instant invention include, but are not limited to, prostate cancer, colorectal cancer, pancreatic cancer, cervical cancer, stomach cancer (gastric cancer), endometrial cancer, brain cancer, glioblastoma, liver cancer, bladder cancer, ovarian cancer, testicular cancer, head and neck cancer, throat cancer, skin cancer, melanoma, basal carcinoma, mesothelioma, lymphoma, leukemia, esophageal cancer, breast cancer, rhabdomyosarcoma, sarcoma, lung cancer, small-cell lung carcinoma, non-small-cell carcinoma, adrenal cancer, thyroid cancer, renal cancer, bone cancer, and choriocarcinoma. In a particular embodiment, the cancer forms a tumor. In a particular embodiment, the cancer is metastatic. In a particular embodiment, the cancer is melanoma (e.g., a melanoma with a mutant BRAF (e.g., BRAF^{V600E})).

Chemotherapeutic agents are compounds that exhibit anticancer activity and/or are detrimental to a cell (e.g., a toxin). Suitable chemotherapeutic agents include, but are not limited to: toxins (e.g., saporin, ricin, abrin, ethidium bromide, diptheria toxin, and Pseudomonas exotoxin); taxanes; alkylating agents (e.g., temozolomide, nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes (e.g., cisplatin, carboplatin, tetraplatin, ormaplatin, thioplatin, satraplatin, nedaplatin, oxaliplatin, heptaplatin, iproplatin, transplatin, and lobaplatin); bioreductive alkylators such as mitomycin, procarbazine, dacarbazine and altretamine); DNA strand-breakage agents (e.g., bleomycin); topoisomerase II inhibitors (e.g., amsacrine, menogaril, amonafide, dactinomycin, daunorubicin, N,N-dibenzyl daunomycin, ellipticine, daunomycin, pyrazoloacridine, idarubicin, mitoxantrone, m-AMSA, bisantrene, doxorubicin (adriamycin), deoxydoxorubicin, etoposide (VP-16), etoposide phosphate, oxanthrazole, rubidazone, epirubicin, bleomycin, and teniposide); DNA minor groove binding agents (e.g., plicamydin); antimetabolites (e.g., folate antagonists such as methotrexate and trimetrexate); pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); anthracyclines; tubulin interactive agents (e.g., vincristine, vinblastine, and paclitaxel (Taxol®)); and antibodies (e.g., HER2 antibodies (e.g., trastuzumab)).

Radiation therapy refers to the use of high-energy radiation from x-rays, gamma rays, neutrons, protons and other sources to target cancer cells. Radiation may be administered externally or it may be administered using radioactive material given internally. Chemoradiation therapy combines chemotherapy and radiation therapy.

In a particular embodiment, the methods of the instant invention (e.g., for treating, inhibiting, and/or preventing cancer or for increasing/improving the therapeutic efficacy of an anti-cancer therapy) comprise administering a polyamine transport system inhibitor (optionally with an ornithine decarboxylase inhibitor) and an immune checkpoint inhibitor (e.g., immune checkpoint blockade therapy). Examples of immune checkpoint inhibitors include, without limitation: PD-1 inhibitors (e.g., antibodies, particularly monoclonal antibodies, immunologically specific for PD-1 such as pembrolizumab (Keytruda®) and nivolumab (Opdivo®)); PD-L1 inhibitors (e.g., antibodies, particularly monoclonal antibodies, immunologically specific for PD-L1 such as atezolizumab (Tecentriq®)); and CTLA-4 inhibitors (e.g., antibodies, particularly monoclonal antibodies, immunologically specific for CTLA-4 such as ipilimumab (Yervoy®)).

In a particular embodiment, the methods of the instant invention (e.g., for treating, inhibiting, and/or preventing cancer or for increasing/improving the therapeutic efficacy of an anti-cancer therapy) comprise administering a polyamine transport system inhibitor (optionally with an ornithine decarboxylase inhibitor) and a cancer vaccine (therapeutic vaccine). A “cancer vaccine” refers to a compound or composition which elicits an immune response against a specific cancer or a variety of cancers. Cancer vaccines include, without limitation: prostatic acid phosphatase (e.g., Sipuleucel-T, Provenge®; e.g., for prostate cancer); heat shock protein gp96 (e.g., oncophage; e.g., for kidney cancer, melanoma, or glioma), epidermal growth factor (EGF) (e.g., CimaVax-EGF; e.g., for lung cancer), Her2/neu (e.g., Neuvenge™, lapuleucel-T; e.g., for breast, colon, bladder, or ovarian cancer), and mucin 1 (e.g., tecemotide, emepepimut-S; e.g., for breast cancer).

In a particular embodiment, the methods of the instant invention (e.g., for treating, inhibiting, and/or preventing cancer or for increasing/improving the therapeutic efficacy of an anti-cancer therapy) comprise administering a polyamine transport system inhibitor (optionally with an ornithine decarboxylase inhibitor) and an adoptive T cell therapy. Adoptive T cell therapy includes the ex vivo expansion of autologous naturally occurring tumor specific T cells which are then transferred back into the cancer patient. Prior to this adoptive transfer, hosts can be immunodepleted by irradiation and/or chemotherapy. As used herein, adoptive T cell therapy can also encompass the ex vivo genetic modification of normal peripheral blood T cells to confer specificity for tumor-associated antigens (e.g., by expressing chimera antigen receptors (CARs) or TCRs of T cells with the desired anti-tumor response). CARs have antibody-like specificities and recognize MHC-nonrestricted structures on the surface of target cells grafted onto the TCR intracellular domains capable of activating T cells.

In accordance with another aspect of the instant invention, methods for treating, inhibiting, and/or preventing a human immunodeficiency virus (HIV) infection in a subject are provided. The method comprises administering a polyamine transport system inhibitor (optionally with an ornithine decarboxylase inhibitor), as described above. The method may further comprise the administration of an antiviral agent, particularly an anti-HIV compound/agent. The therapies may be administered consecutively (before or after) and/or at the same time (concurrently) as the polyamine transport system inhibitor (optionally with an ornithine decarboxylase inhibitor). Co-administered agents may be administered in the same composition or in separate compositions.

An anti-HIV compound or an anti-HIV agent is a compound which inhibits HIV (e.g., replication or infection). Examples of an anti-HIV agent include, without limitation:

(I) Nucleoside-analog reverse transcriptase inhibitors (NRTIs). NRTIs refer to nucleosides and nucleotides and analogues thereof that inhibit the activity of HIV-1 reverse transcriptase. An example of nucleoside-analog reverse transcriptase inhibitors is, without limitation, adefovir dipivoxil.

(II) Non-nucleoside reverse transcriptase inhibitors (NNRTIs). NNRTIs are allosteric inhibitors which bind reversibly at a nonsubstrate-binding site on the HIV reverse transcriptase, thereby altering the shape of the active site or blocking polymerase activity. Examples of NNRTIs include, without limitation, delavirdine, efavirenz, nevirapine, capravirine, emivirine (+)-calanolide A and B, etravirine, rilpivirne, and dapivirine.

(III) Protease inhibitors (PI). Protease inhibitors are inhibitors of the HIV-1 protease. Examples of protease inhibitors include, without limitation, darunavir, amprenavir, tipranivir, indinavir, saquinavir, fosamprenavir, lopinavir, ritonavir, atazanavir, nelfinavir, lasinavir, and brecanavir.

(IV) Fusion or entry inhibitors. Fusion or entry inhibitors are compounds, such as peptides, which act by binding to HIV envelope protein and blocking the structural changes necessary for the virus to fuse with the host cell. Examples of fusion inhibitors include, without limitation, CCR5 receptor antagonists (e.g., maraviroc), enfuvirtide, T-20 (DP-178) and T-1249.

(V) Integrase inhibitors. Integrase inhibitors are a class of antiretroviral drug designed to block the action of integrase, a viral enzyme that inserts the viral genome into the DNA of the host cell. Examples of integrase inhibitors include, without limitation, raltegravir, elvitegravir, cabotegravir, dolutegravir, and MK-2048.

Anti-HIV compounds also include maturation inhibitors (e.g., bevirimat). Maturation inhibitors are typically compounds which bind HIV gag and disrupt its processing during the maturation of the virus. Anti-HIV compounds also include HIV vaccines such as, without limitation, ALVAC® HIV (vCP1521), AIDSVAX®B/E (gp120), and combinations thereof. Anti-HIV compounds also include HIV antibodies (e.g., antibodies against gp120 or gp41), particularly broadly neutralizing antibodies.

More than one anti-HIV agent may be used, particularly where the agents have different mechanisms of action (as outlined above). In a particular embodiment, the anti-HIV therapy is highly active antiretroviral therapy (HAART).

In accordance with another aspect of the instant invention, methods for administering a vaccine to a subject are provided. The method comprises administering a polyamine transport system inhibitor (optionally with an ornithine decarboxylase inhibitor), as described above, in combination with a vaccine. In other words, the polyamine transport system inhibitor (optionally with an ornithine decarboxylase inhibitor) is used as an adjunct therapy. For example, the polyamine transport system inhibitor (optionally with an ornithine decarboxylase inhibitor) can be administered in combination with a vaccine such as, without limitation, a hepatitis B (HepB) vaccine, rotavirus vaccine, diphtheria, pertussis, and tetanus vaccine, Haemophilus influenzae type b (Hib) vaccine, pneumococcal vaccine, polio vaccine, influenza vaccine, measles, mumps, and rubella (MMR) vaccine, varicella vaccine, hepatitis A vaccine, meningococcus vaccine, and human papillomavirus vaccine. The therapies may be administered consecutively (before or after) and/or at the same time (concurrently) as the polyamine transport system inhibitor (optionally with an ornithine decarboxylase inhibitor). Co-administered agents may be administered in the same composition or in separate compositions.

In accordance with another aspect of the instant invention, compositions are provided comprising one or more of the above identified agents and a pharmaceutically acceptable carrier. In a particular embodiment, when the agents are contained in separate compositions as described above, the separate compositions are contained within a kit.

The compositions of the present invention can be administered by any suitable route, for example, by injection (e.g., for local (direct, including to or within a tumor) or systemic administration), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intrapulmonary, intraareterial, intrarectal, intramuscular, and intranasal administration. In a particular embodiment, the composition is administered to the blood (e.g., intravenously). In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention (e.g., Remington: The Science and Practice of Pharmacy). The pharmaceutical composition of the present invention can be prepared, for example, in liquid form, or can be in dried powder form (e.g., lyophilized for later reconstitution).

As used herein, “pharmaceutically acceptable carrier” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the molecules to be administered, its use in the pharmaceutical preparation is contemplated.

The dose and dosage regimen of the molecule of the invention that is suitable for administration to a particular patient may be determined by a physician considering the patient's age, sex, weight, general medical condition, and the specific condition and severity thereof for which the inhibitor is being administered. The physician may also consider the route of administration, the pharmaceutical carrier, and the molecule's biological activity.

Selection of a suitable pharmaceutical preparation depends upon the method of administration chosen. For example, the molecules of the invention may be administered by direct injection into any cancerous tissue or into the area surrounding the cancer. In this instance, a pharmaceutical preparation comprises the molecules dispersed in a medium that is compatible with the cancerous tissue.

Molecules of the instant invention may also be administered parenterally by intravenous injection into the blood stream, or by subcutaneous, intramuscular, intrathecal, or intraperitoneal injection. Pharmaceutical preparations for parenteral injection are known in the art. If parenteral injection is selected as a method for administering the molecules, steps should be taken to ensure that sufficient amounts of the molecules reach their target cells to exert a biological effect. The lipophilicity of the molecules, or the pharmaceutical preparation in which they are delivered, may have to be increased so that the molecules can arrive at their target locations. Methods for increasing the lipophilicity of a molecule are known in the art.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous, oral, topical, or parenteral. In preparing the molecule in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form in which case solid pharmaceutical carriers are obviously employed. If desired, tablets may be sugar-coated or enteric-coated by standard techniques. For parenterals, the carrier will usually comprise sterile water, though other ingredients, for example, to aid solubility or for preservative purposes, may be included. Injectable suspensions may also be prepared, in which case appropriate liquid carriers, suspending agents and the like may be employed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art. The appropriate dosage unit for the administration of the molecules of the instant invention may be determined by evaluating the toxicity of the molecules in animal models. Various concentrations of pharmaceutical preparations may be administered to mice with transplanted human tumors, and the minimal and maximal dosages may be determined based on the results of significant reduction of tumor size and side effects as a result of the treatment. Appropriate dosage unit may also be determined by assessing the efficacy of the treatment. The dosage units of the molecules may be determined individually or in combination with each anti-cancer therapy according to greater shrinkage and/or reduced growth rate of tumors.

The pharmaceutical preparation comprising the molecules of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

### Definitions

The following definitions are provided to facilitate an understanding of the present invention:

The singular forms “a,” “an,” and “the” include plural referents unless the context clearly dictates otherwise.

As used herein, the terms “host,” “subject,” and “patient” refer to any animal, including mammals such as humans.

A “therapeutically effective amount” of a compound or a pharmaceutical composition refers to an amount effective to prevent, inhibit, treat, or lessen the symptoms of a particular disorder or disease.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, excipient, auxilliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described, for example, in “Remington's Pharmaceutical Sciences” by E. W. Martin.

An “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “kit” generally refers to a collection of elements that together are suitable for a defined use. In a particular embodiment, a “kit” refers to a collection of containers containing the necessary compositions to carry out the method of the invention, typically in an arrangement both convenient to the user and which ensures the chemical stability of the compositions. The kit may comprise a delivery system having one or more containers (such as tubes or vials). For example, such delivery systems include systems (e.g., enclosures (e.g., boxes)) that allow for the storage, transport, or delivery of the compositions of the instant invention and/or supporting materials (e.g., instruction material) from one location to another.

The following examples are provided to illustrate various embodiments of the present invention. The examples are not intended to limit the invention in any way.

### Example 1

### Materials and Methods

### Animals

Female C57B16 or Balb/C Mice were Obtained from Charles Rivers/NCI.

### Cell Culture

B16F10-sTAC cells engineered to express SIINFEKL (SEQ ID NO: 1) peptide were cultured in DMEM supplemented with 10% fetal bovine serum and 1× Penicillin/Streptomycin. CT26.CL25 cells were cultured in DMEM supplemented with 10% fetal bovine serum, 1× penicillin/streptomycin and 0.8 mg/ml of G418 disulfate (Fisher Scientific). CT26.CL25 (American Type Culture Collection, Rockville, Md.) is a subclone of CT26 colon carcinoma cells that have been transduced with Escherichia coli β-gal gene, which have been shown to be equally as lethal as the parental clone CT26.WT, in normal mice.

### In Vivo Tumor Models

Tumor models were established by subcutaneous injections of 5×10⁵ B16F10-sTAC cells in C57B16 mice or 5×10⁵ CT26.CL25 cells in Balb/c mice. Mice were monitored twice a week for tumor growth. Treatment with 0.25% (w/v) DFMO in the drinking water and the Trimer PTI (3 mg/kg daily by intraperitoneal injection) was initiated when tumors were palpable (50-100 mm³). For adoptive transfers, spleens were excised and pressed through a nylon filter to obtain a single cell suspension. The resulting suspension was incubated with red cell lysis buffer for 5 minutes to lyse red blood cells. Gr1⁺ cells were stained with a PE conjugated antibody and then isolated using anti-PE microbeads (Miltenyi Biotec) as per the manufacturer's instructions. Gr1⁺ positive cells were injected into the retro-orbital cavity. Tumor growth was assessed morphometrically using calipers, and tumor volumes were calculated using the formula V (mm³)=π/6×A×B² (A is the larger diameter and B is the smaller diameter).

### Antigen-Specific T-Cell Response Detection by IFN-γ ELISpot

Upon sacrifice, splenocytes from B16F10-sTAC tumor bearing mice and tumor cell suspensions from CT26.CL25 tumor bearing mice were analyzed for IFN-γ producing cells by enzyme-linked immunosorbent spot (ELISpot) assay. Multiscreen filtration plates (Millipore) were coated with 0.5 μg/mL of purified anti-mouse IFN-γ capture antibody (Biolegend) overnight at 4° C. Single-cell suspensions of splenocytes or tumors were plated at 1×10⁶ and 5×10⁵ per well respectively. For ELISpot assays with splenocytes from B16F10-sTAC tumor bearing mice, cells were stimulated with the SIINFEKL (SEQ ID NO: 1) peptide (Anaspec) at 20 jag/mL. Cells from CT26.CL25 tumors were treated with a known H-2D-restricted 3-galactosidase peptide (TPHPARIGL (SEQ ID NO: 2); ChemPep). After 16 hours of stimulation at 37° C., the cells were removed by washing and spots were developed with a biotinylated anti-IFN-γ detection antibody and streptavidin-horseradish peroxidase conjugate followed by NITRO-blue tetrazolium chloride and 5-bromo-4-chloro-3′-indoylphosphate p-toluidine salt substrate (Sigma). Spot numbers were counted, and data were reported as IFN-γ-spot forming cells per 10⁶ cells.

### Immunohistochemistry

Mouse tumor tissues were fixed in 4% paraformaldehyde in PBS overnight and embedded in paraffin. Sections were deparaffinized, hydrated, and then heated in 0.01 mol/L sodium citrate buffer (pH 6.0) in a steamer for 8 minutes. Sections were incubated with the primary antibody (rat monoclonal anti-mouse F4/80 [BioRad MCA497GA]) for 2 hours at room temperature followed by biotinylated secondary and then an avidin HRP complex (Vectastain Elite ABC KIT, Vector Laboratories, INC). Immunoreactive cells were localized by incubating the sections with diaminobenzidine and peroxide and then counterstaining with hematoxylin. Pictures were obtained using a Zeiss Axiophot microscope (Carl Zeiss Inc.) with a digital color camera and corresponding software.

### Cytokine Analysis

B16F10-sTAC tumors were flash frozen in liquid nitrogen, ground and homogenized in PBS containing 0.5 μM dithiothreitol, 0.5 μM Pefabloc and 8 μg/mL leupeptin. Samples were analyzed for monocyte chemoattractant protein-1 (MCP-1), interleukin-6 (IL-6). IL-10, tumor necrosis factor alpha (TNF-α) and interferon gamma (IFN-γ) using the Mouse Inflammation Cytometric Bead Array reagents (BD Biosciences, San Jose, Calif.) and flow cytometry as per the manufacturer's protocol. VEGF cytokine levels were analyzed using the mouse VEGF Quantikine ELISA (R&D systems) as per the manufacturer's instructions.

### Flow Cytometry Analysis of Immune Cell Infiltrates

Tumor tissue was digested in a 0.3% collagenase/0.1% hyaluronidase solution, pressed through a nylon mesh filter to obtain a single cell suspension and incubated in red cell lysis buffer (0.17 M Tris-HCL, 0.16 M NH₄Cl) for 5 minutes. Cells were spun down and resuspended in a 40% Percoll solution (GE Healthcare). The tumor cell suspension was then underlaid with an 80% Percoll solution and spun at 325×g for 23 minutes. Cells at the interface between the 40% and 80% Percoll solutions were removed, washed and prepared for flow cytometric analysis. Equal numbers of viable cells were stained with combinations of the following: CD8a-PECy7, CD4-PE, F4/80-PECy7, CD206-FITC, Gr1-APC, CD1 b-PE, CD45-APC-Cy7, Granzyme B-FITC and IFN-γ-APC. Flow-cytometric data were acquired on a BD FACSCanto II cytometer and analyzed using FACSDiva software (BD Biosciences).

### Statistical Analysis

All in vitro experiments were performed at least in triplicate, and data were compiled from two to three separate experiments. Analyses were done using a 1-way ANOVA with a Tukey test for statistical significance or a Students t-test. In vivo studies were carried out using multiple animals (n=5-10 per treatment group). Tumor growth curves were analyzed with a Generalized Linear model with fixed effects of treatment and time. Data were examined for the interaction between treatment groups and day of observations, testing whether the slopes of the growth curves (tumor volume vs. day of observation) were significantly different for the control and treatment groups. In all cases, values of p<0.05 were regarded as being statistically significant.

### Results

### PBT Therapy Reduces Tumor Growth and Progression

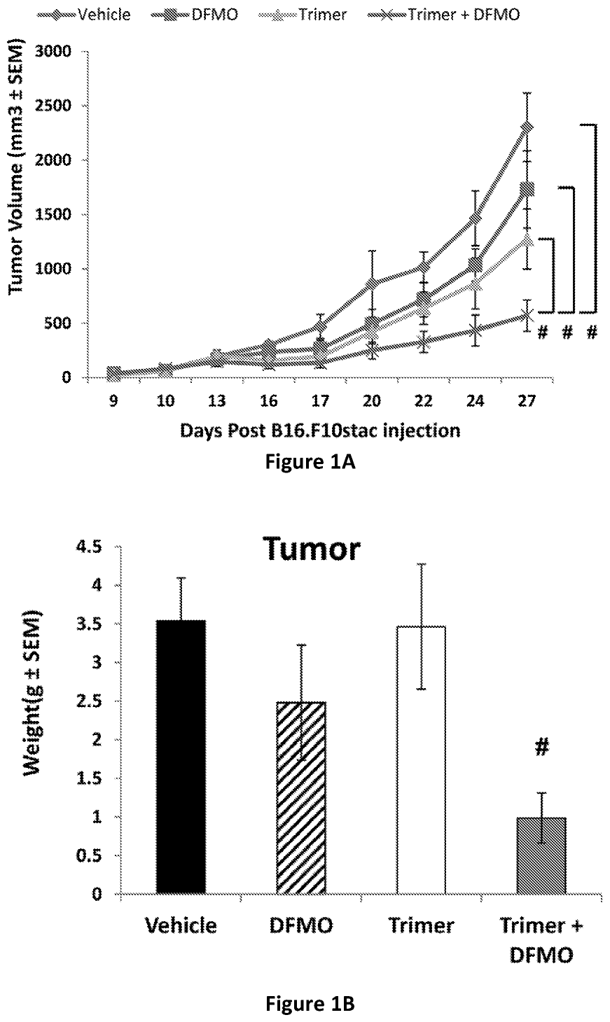

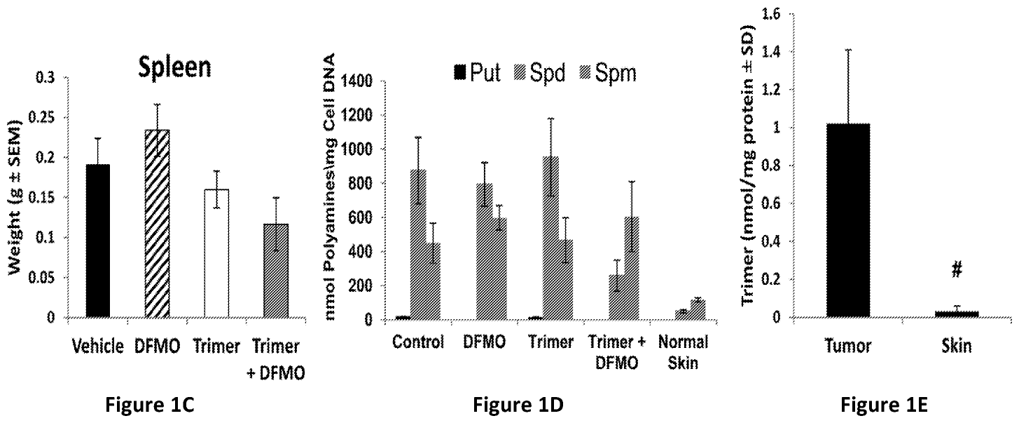

To evaluate the effect of polyamine blockade therapy (PBT), the B16F10-sTAC melanoma model was used. Following subcutaneous injection of B16F10-sTAC cells expressing SIINFEKL (SEQ ID NO: 1) peptide in C57/B16 mice, treatment was initiated when tumors were between 50-100 mm³ in size. Mice were administered Trimer PTI (N1, N1′, N1″-(benzene-1,3,5-triyltris(methylene))tris(N4-(4-(methylamino) butyl)butane-1,4-diamine) (i.p. injection 3 mg/kg daily), with or without 0.25% α-difluoromethylornithine (DFMO, an ODC inhibitor) (w/v) in the drinking water. Treatment with either DFMO or Trimer PTI individually only modestly reduced B16F10-sTAC tumor growth compared to vehicle treatment (FIGS. 1A and 1B). However, there was a significant inhibitory effect on tumor growth in mice treated with both DFMO and the Trimer PTI with a 4-fold reduction in final tumor weight compared to vehicle treated mice. Treatment with Trimer PTI with or without DFMO had no significant effect on spleen weight (FIG. 1C). High-performance liquid chromatography (HPLC) analysis of the polyamine content in tumors showed that all polyamines, including putrescine, spermidine, and spermine, were elevated in the tumors compared to non-tumor bearing skin. Treatment with DFMO alone reduced the levels of putrescine compared to vehicle-treated mice, whereas treatment with Trimer PTI alone had no discernible effect on polyamine levels (FIG. 1D). However, co-treatment with both DFMO and the Trimer PTI significantly reduced the levels of both putrescine and spermidine in the tumors compared with vehicle treated mice (FIG. 1D). Mass spectrometry analysis demonstrated that the Trimer PTI accumulated preferentially in the tumor (30 fold) of PBT-treated mice compared to levels detected in surrounding non-tumor bearing skin (FIG. 1E).

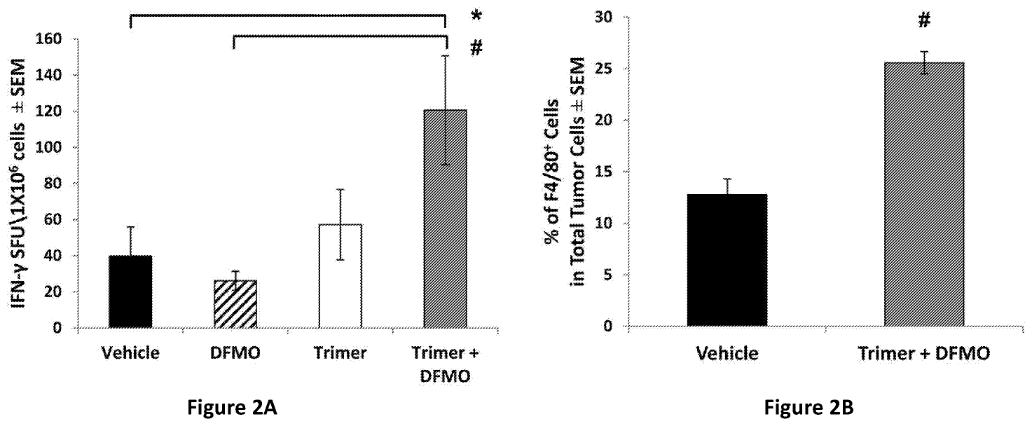

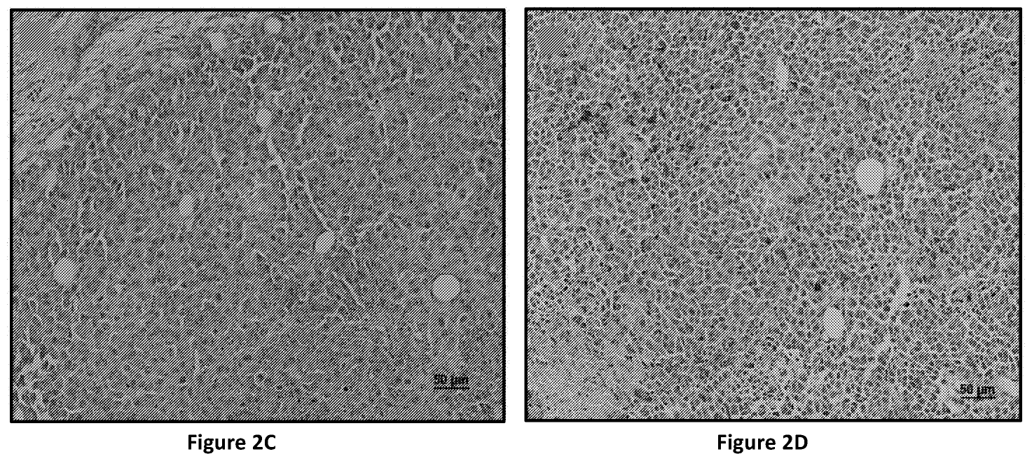

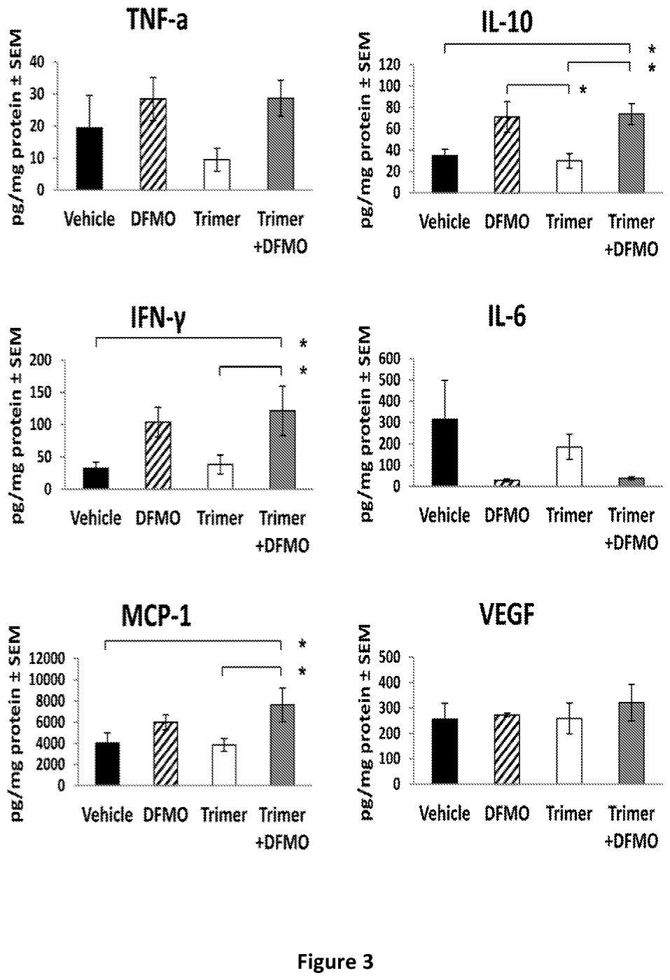

The decreased tumor growth in mice treated with DFMO and Trimer PTI was associated with a significant increase in the number of IFN-γ producing splenocytes as measured by the ELISpot assay following ex vivo stimulation with SIINFEKL (SEQ ID NO: 1) peptide (FIG. 2A) as well as an increase in the number of F4/80 positive macrophages in the tumor (FIG. 2B). The increase in F4/80 positive macrophages in the tumors of mice co-treated with DFMO and Trimer PTI was also associated with movement of the macrophages from the periphery of the tumor, as seen in vehicle treated tumors to the interior (FIGS. 2C and 2D). The increased tumor infiltration of macrophages correlated with increased levels of proinflammatory cytokines including monocyte chemoattractant protein-1 (MCP-1) which is one of the key chemokines that regulate migration and infiltration of monocytes/macrophages. Treatment with DFMO or Trimer PTI individually did not significantly alter cytokine levels compared to vehicle treated mice (FIG. 3). However, co-treatment with both DFMO and Trimer PTI significantly increased the levels of IL-10, IFN-γ, and MCP-1, i.e., cytokines associated with increased immune activity in the tumor and tumor microenvironment.

### Anti-Tumor Effects of PBT Rely on Host Immune Processes

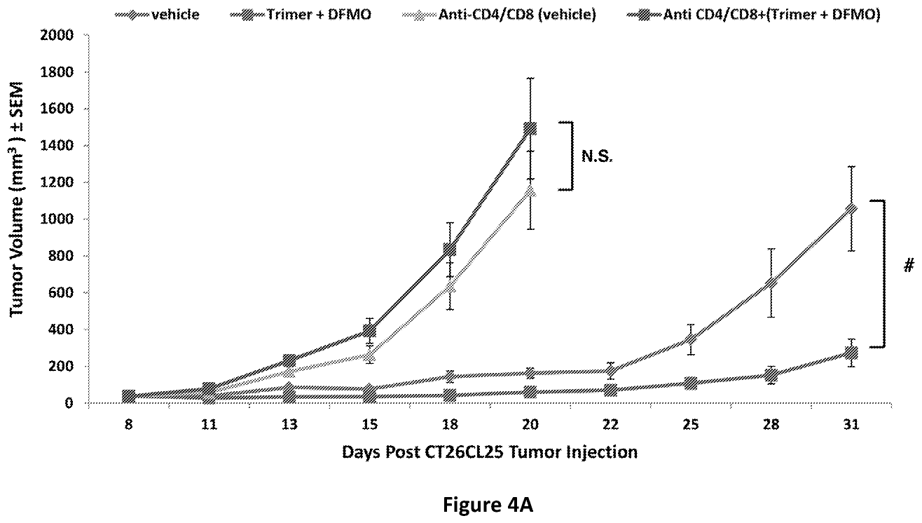

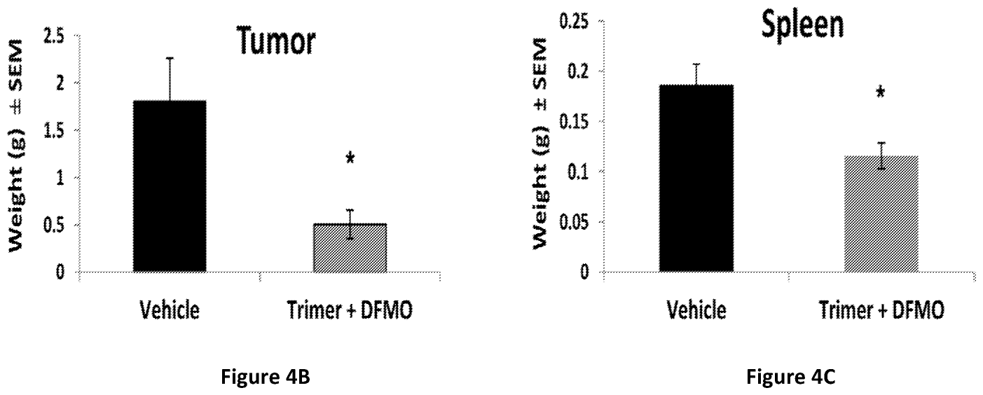

Because decreased tumor growth in mice co-treated with DFMO and Trimer PTI was associated with increased T-cell IFN-γ production as measured by ELISpot assay as well as increased levels of IL-10, MCP-1 and IFN-γ, it was hypothesized that PBT anti-tumorigenic properties may be dependent on host immune system competence. To test this, mice with subcutaneous CT26.CL25 colon carcinoma tumors were treated with anti-CD4 and anti-CD8 antibodies to deplete CD4⁺ and CD8⁺ T-cells before and during the treatment with PBT. As expected, tumors in mice treated with anti-CD4/CD8 antibodies grew at an accelerated rate compared to vehicle treated mice (FIG. 4A). Whereas PBT treatment significantly reduced CT26.CL25 tumor growth in mice possessing a full complement of CD4+ and CD8⁺ T-cells (FIGS. 4A and 4B), PBT treatment had no significant inhibitory effects on tumor growth in mice that had received anti-CD4/CD8 antibodies (FIG. 4A). The spleen weight of CT26.CL25 tumor bearing mice was significantly higher compared to those on PBT treatment and mice without tumors (FIG. 4C).

PBT Treatment Reduces Specific Subpopulations of Suppressor and Pro-Tumorigenic Cells while Increasing T Effector Cell Interferon-y and Granzyme B Production

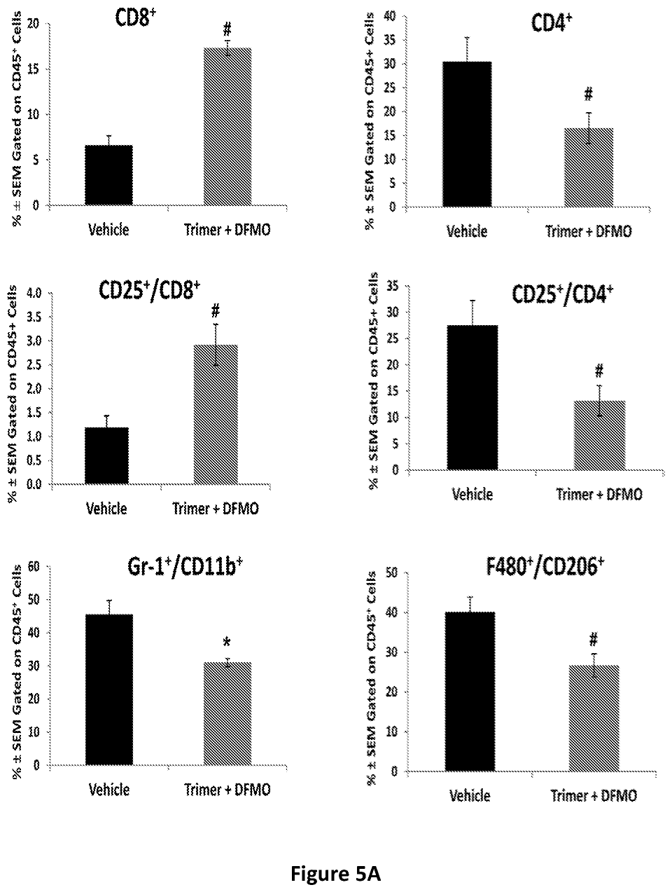

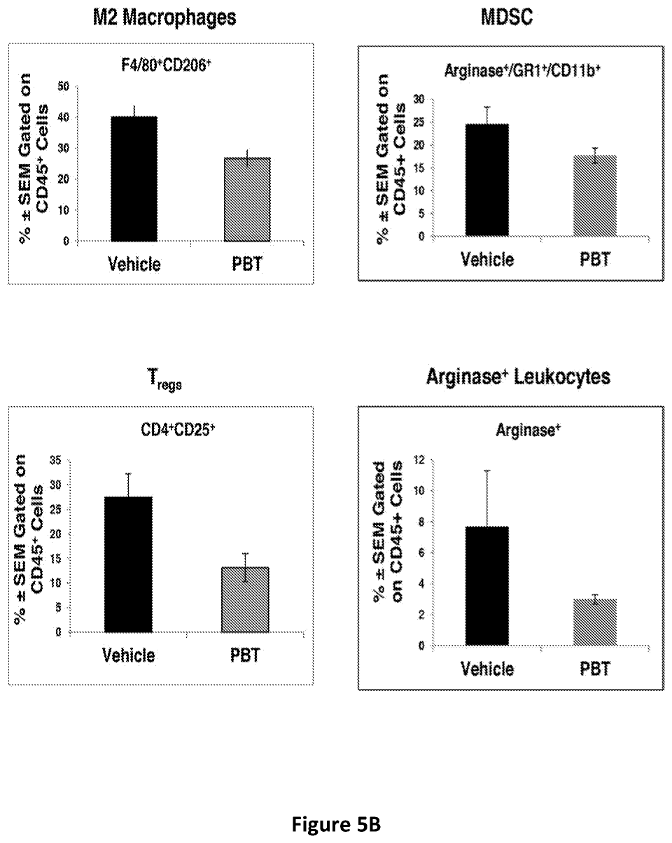

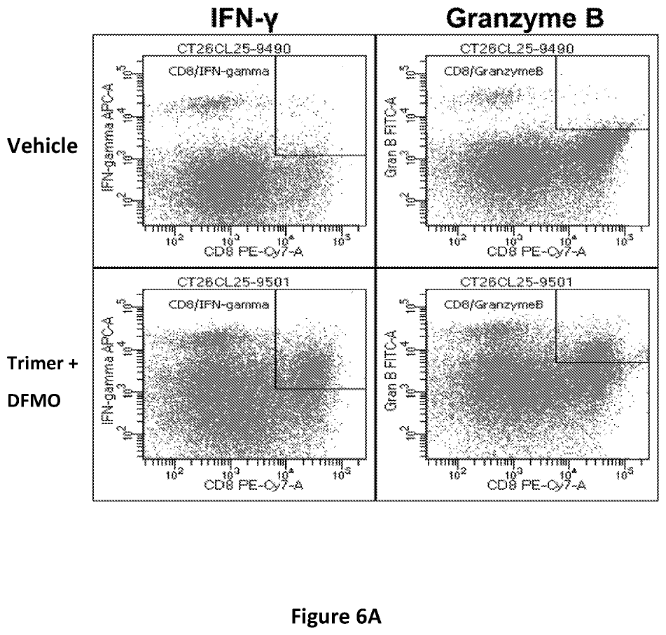

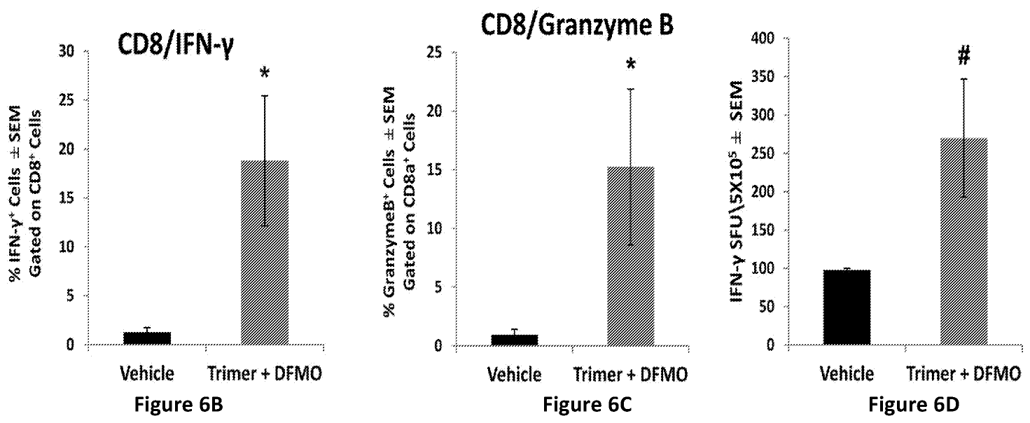

Subpopulations of immune cell infiltrates in CT26.CL25 tumors were analyzed by flow cytometry following isolation of tumor leukocytes using a discontinuous Percoll gradient. Treatment with PBT significantly decreased the populations of Gr1⁺ CD11b⁺ myeloid derived suppresser cells (MDSC) and CD25⁺ CD4⁺ T regulatory cells (Tregs), major immunosuppressive cell types found in many different tumor types (FIG. 5A). PBT treatment also significantly reduced the levels of F4/80+CD206⁺ M2 macrophages that have been shown to support and promote tumor growth in a broad range of tumors. Similar changes were also noted in the FACS analyses of splenocytes from vehicle and PBT treated mice. Treatment with PBT significantly reduced M2 macrophages (F4/80⁺ CD206⁺), MDSCs (arginase⁺/Gr1⁺/CD11b⁺) and Tregs (CD4⁺ CD25⁺) populations as well as overall arginase⁺ CD45⁺ leukocytes (FIG. 5B). While PBT treatment reduced the levels of immunosuppressive and pro-tumorigenic leukocytes that infiltrated the tumors, it also significantly increased the percentage of CD8⁺ cytotoxic T-cells in the tumors (FIG. 5A) which are immune cells responsible for directly killing tumors cells. To further characterize CD8⁺ T effector cells infiltrating the tumors, the intracellular levels of IFN-γ and granzyme B, proteins that are released from cytotoxic T-cells to stimulate the immune system and to actively induce apoptosis in target cells, respectively, were analyzed. Compared to vehicle-treated mice, treatment with PBT significantly increased the percentage of CD8⁺ T-cells producing IFN-γ from 1.3+0.3% to 18.8+6.6% (FIGS. 6A and 6B), while increasing the percentage of CD8⁺ T-cells producing granzyme B to 15.2+5.4% from 1.0+0.3% for vehicle treated mice (FIGS. 6A and 6C). Isolated infiltrating leukocytes from the tumors were also assayed in an IFN-γ ELISpot assay. Treatment with PBT was associated with a significant increase in the number of IFN-γ producing isolated infiltrating leukocytes following ex vivo stimulation with the CT26.CL25 tumor-specific LacZ peptide (FIG. 6D).

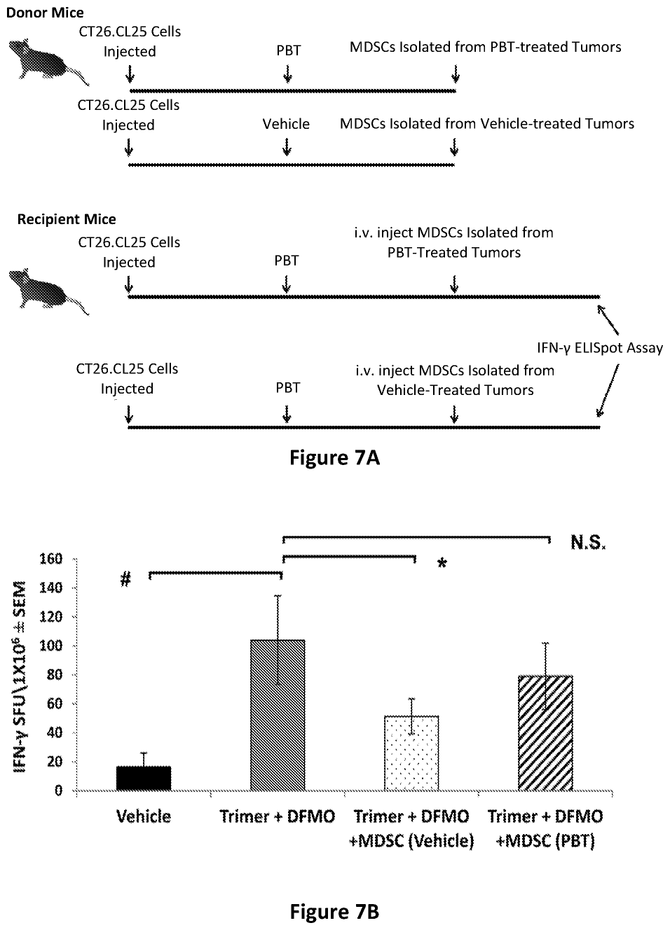

Because treatment with PBT significantly reduced MDSCs and Tregs, while increasing cytotoxic T-cell activity, it was hypothesized that PBT treatment was directly effecting suppressor cell populations, which subsequently lessened suppression of CD8⁺ T-cells and increased T-cell activity (as seen by the IFN-γ ELISpot assays). To test this hypothesis, MDSCs were isolated from CT26.CL25 tumor bearing mice that were vehicle control treated or treated with PBT, and then MDSCs were adoptively transferred to recipient CT26.CL25 tumor-bearing mice treated with PBT, one week before they were sacrificed (FIG. 7A). The single adoptive transfer of MDSCs from vehicle treated mice into recipient tumor-bearing mice treated with PBT blunted the PBT-induced increase in antigen-specific T-cell IFN-γ response (FIG. 7B). However, the adoptive transfer of MDSCs isolated from tumor-bearing, PBT-treated mice into recipient tumor-bearing mice treated with PBT resulted in no significant reduction in tumor-specific T-cell IFN-γ production compared to that seen with PBT treated mice that did not receive a MDSC adoptive transfer. These data indicate that PBT treatment had modified or altered the MDSCs making them less immunosuppressive. Overall these results indicate that co-treatment with DFMO and Trimer PTI significantly inhibits tumor growth in multiple tumor models by reversing the immunosuppressive tumor microenvironment.

An increased need for polyamines is essential for oncogenic activity in tumors, both in providing for biomass via dramatic increases in protein translation and in contributing to survival pathways for the tumor cells. Elevated intracellular polyamine levels in tumors are achieved by induction of polyamine biosynthesis enzymes and import pathways. The development of polyamine transport inhibitors is important since many tumor types upregulate PTS activity in the presence of polyamine biosynthesis inhibitors such as DFMO. It is shown herein that the combination PBT treatment with DFMO and the novel Trimer PTI significantly lowers intracellular tumor polyamine levels resulting in reduced tumor growth in animals. Importantly, the results demonstrate that PBT is an anti-metabolic treatment that reverts tumor-induced immunosuppression by re-conditioning the tumor microenvironment. Indeed, PBT reduction of tumor myeloid suppressor cell populations and activation of antitumor immune responses is important for its anti-tumor efficacy since it fails to decrease tumor growth in mice lacking T cells.

Polyamines are important modulators of the immune response, particularly in the tumor microenvironment where they are found in high concentrations. Spermine inhibits the production of IL-12, TNF-α, IL-1, IL-6, MIP-1a, and MIP-1b by in vitro-stimulated macrophages (Hasko et al. (2000) Shock 14:144-9; Zhang et al. (2000) Crit. Care Med., 28:N60-6) and protects against lethal sepsis by attenuating local and systemic inflammatory response (Zhu et al. (2009) Mol. Med., 15:275-82). In vitro studies have also shown that polyamines suppress lymphocyte proliferation, decrease macrophage tumoricidal activity and neutrophil motility, and decrease IL-12-dependent NK cell activity (Labib et al. (1981) Eur. J. Immunol., 11:266-9; Ferrante et al. (1986) Int. J. Immunopharmacol., 8:411-7; Chamaillard et al. (1997) Br. J. Cancer 76:365-70; Chamaillard et al. (1993) Anticancer Res., 13:1027-33; Soda, K. (2011) J. Exp. Clin. Cancer Res., 30:1-9). Significantly elevated IL-10 levels in tumors of PBT treated mice were observed. IL-10 is a highly pleiotropic cytokine that has been characterized as both a tumor promoter and an inhibitor of tumor progression depending on context. Multiple studies have found a positive correlation between IL-10 levels and poor prognosis in melanoma, likely due to immunosuppressive properties of IL-10 (Mannino et al. (2015) Cancer Lett., 367:103-7). However, it has been found that IL-10 has potent anti-tumor effects. Intravenous administration of IL-10 results in the rejection of implanted tumors due to activation and expansion of resident CD8⁺ T-cells (Emmerich et al. (2012) Cancer Res., 72:3570-81). An increase in IFN-γ and granzyme B expression along with a three-fold increase in tumor-infiltrating cytotoxic leukocytes in mice receiving IL-10 has also been observed (Emmerich et al. (2012) Cancer Res., 72:3570-81). Herein, treatment with DFMO alone or Trimer alone was not sufficient to significantly inhibit tumor growth. However, blocking both polyamine biosynthesis and polyamine cellular uptake with PBT not only decreased tumor polyamine levels resulting in significantly reduced tumor growth but also enhanced an anti-tumor immune response. In mice with either B16F10 or CT26.CL25 tumors, splenocytes from PBT-treated mice demonstrated a significant increase in tumor-specific IFN-γ expression that was not present in tumor-bearing mice treated with DFMO or Trimer alone. The data shows that PBT stimulates an anti-tumor immune effect that is T-cell dependent since the anti-tumor effect of PBT was lost in mice where CD4⁺ and CD8⁺ T cells were antibody-depleted. PBT anti-tumor activity was accompanied by an increase in activated CD8⁺ T-cells and a decrease in immunosuppressive tumor infiltrating cells including Gr-1+CD11b myeloid derived suppressor cells (MDSCs), CD4+CD25⁺ Tregs, and CD206+F4/80⁺ M2 macrophages. The anti-tumor activity of PBT combination treatment deprives polyamine-addicted tumors of polyamines that they need for survival and also relieves polyamine-mediated immunosuppression in the tumor microenvironment.

The Trimer PTI is a competitive inhibitor of the PTS that out-competes native polyamines for binding to the cell surface receptors (e.g. heparan sulfate proteoglycans) of the PTS (Phanstiel et al. (2007) Amino Acids 33:305-13; Poulin et al. (2012) Amino Acids 42:711-23). Normal cells can synthesize sufficient levels of polyamines and have very low PTS activity compared to tumor cells that have a much greater need for polyamines. Mass spec analysis shows that the high PTS activity in tumor cells results in higher accumulation of Trimer PTI in tumors compared to normal tissue, thus reducing toxicity in non-tumor tissues while more specifically reducing polyamine levels in the tumor. Another polyamine-based PTI, AMXT1501, has also been shown to have anti-tumor efficacy when combined with DFMO (Hayes et al. (2014) Cancer Immunol. Res., 2:274-85). However, the design of the lipophilic linear palmitic acid-lysine-spermine structure of AMXT1501 differs from the 1,3,5-tri-substituted benzene of the Trimer PTI (Muth et al. (2014) J. Med. Chem., 57:348-63). For example, the Trimer PTI is more water-soluble, is orally available, and presents three homospermidine ‘messages’ to the receptor compared to the N1-acylated spermine motif presented by AMXT1501 (Muth et al. (2014) J. Med. Chem., 57:348-63). Since the N¹-position in AMXT1501 is N-acylated, this design converts the spermine headgroup into a modified N-alkylated spermidine motif. The homospermidine motif in the Trimer PTI has been shown in vitro to outperform the spermidine tail available in AMXT1501 in terms of selectively targeting cells with active polyamine transport (Muth et al. (2014) J. Med. Chem., 57:348-63; Wang et al. (2003) J. Med. Chem., 46:2663-71; Wang et al. (2003) J. Med. Chem., 46:2672-82). While both PTI approaches have their merits, the N-methylation of the terminal primary amines of the Trimer PTI provides additional metabolic stability to amine oxidases (Kaur et al. (2008) J. Med. Chem., 51:2551-60), and blood and tissue analyses have revealed that its metabolism via demethylation creates an even more potent PTI (Muth et al. (2014) J. Med. Chem., 57:348-63). In summary, the Trimer PTI possesses a balance of properties that include low toxicity, high potency, improved targeting, and metabolic stability.

PBT-activation of a tumor immune response may be due to a direct activating effect on T-cell tumor infiltrates as well as indirect inhibitory effects on myeloid cell subpopulations that suppress T-cell activation. A variety of tumor-infiltrating immunosuppressive myeloid populations, including MDSCs, M2 macrophages, and Treg populations, have been shown to suppress cytotoxic T-cell activity. The data show that PBT decreases levels of multiple immunosuppressive myeloid cell populations that infiltrate tumors. In contrast, another study reported that treatment with higher doses of DFMO alone did not decrease MDSC accumulation in tumor-bearing mice even though DFMO-treatment of MDSCs in short-term culture inhibited their suppression of T-cell proliferation in an arginase-dependent manner (Ye et al. (2016) J. Immunol., 196:915-23). Thus, it appears that polyamine depletion with both DFMO and the Trimer PTI decreases MDSC accumulation in tumor-bearing animals. This is further reinforced with the experimental data showing that adoptive transfer of MDSCs reduced the tumor-specific T-cell IFN-γ production that was stimulated in PBT-treated tumor-bearing mice. However, adoptive transfer of MDSCs from PBT-treated mice did not significantly reduce this tumor-specific T-cell IFN-γ production, indicating that blocking both polyamine biosynthesis and transport in vivo impairs the accumulation and immunosuppressive function of MDSCs. Although the molecular basis for PBT-mediated inhibitory effects on myeloid immunosuppressive activity remains to be determined, DFMO blocks IL-4 induction of arginase activity in macrophages (Hayes et al. (2014) Cancer Immunol. Res., 2:274-85) as well as impairing the immunosuppressive activity of MDSCs via reducing their arginase activity (Ye et al. (2016) J. Immunol., 196:915-23). PBT reduces arginase activity that is induced in not only immunosuppressive myeloid cells but also in tumor epithelial cells with elevated polyamine levels (Hayes et al. (2014) Cancer Immunol. Res., 2:274-85). Furthermore, polyamines released by MDSCs can confer an indoleamine 2,3-dioxygenase 1 (IDO1)-dependent, immunosuppressive phenotype on dendritic cells (Mondanelli et al. (2017) Immunity 46:233-44). Thus, PBT likely activates an anti-tumor immune response via multiple mechanisms affecting the metabolism of both tumor epithelial cells as well as immunosuppressive tumor-associated cell populations.

These data show that combined treatment with both DFMO and the Trimer PTI not only deprives polyamine-addicted tumor cells of polyamines, but also relieves polyamine-mediated immunosuppression in the tumor microenvironment, thus allowing the activation of tumoricidal T-cells. Tumor synthesis and release of polyamines contributes to immune editing of tumors and the selection of immunosuppressive cells in the tumor microenvironment. This polyamine blocking therapy can be used as adjunct cancer treatment both with conventional chemotherapeutic agents and in stimulating anti-tumor immune responses with tumor immunotherapies.

### Example 2

Melanoma is a highly aggressive tumor with poor prognosis in the metastatic stage. Multiple oncogenic mutations (including BRAF, NRAS, KIT) drive this highly heterogeneous disease, with BRAF mutations detected in half of all melanoma tumors (Davies, et al. (2002) Nature 417 (6892):949-954). The treatment of metastatic melanoma has been revolutionized over the last decade with the discovery of highly prevalent BRAF mutations, which drive constitutive activation of the RAS-RAF-MEK-ERK pathway and promote uncontrolled proliferation (Davies, et al. (2002) Nature 417 (6892):949-954). Ninety percent of reported BRAF mutations result in substitution of glutamic acid for valine at amino acid 600 (the V600E mutation) (Solit, et al. (2011) N. Engl. J. Med., 364 (8):772-774; Haq, et al. (2013) Pigment Cell Melanoma Res., 26 (4):464-469). The subsequent rapid development of selective inhibitors of V600E-mutant BRAF proteins (vemurafenib and dabrafenib) demonstrated a major advance in the treatment of melanoma patients harboring the BRAF^{V600E} mutation. However, nearly 100% of the patients exhibit disease progression within 7 months after treatment with BRAF inhibitors (Flaherty, et al. (2010) N. Engl. J. Med., 363(9):809-819; Sosman, et al. (2012) N. Engl. J. Med., 366(8):707-714; Hauschild, et al. (2012) Lancet 380(9839):358-365). Thus, new ways to overcome the acquired resistance to these inhibitors are urgently needed to increase survival in melanoma patients.

An alternative approach is to target a downstream pathway that is essential for survival of oncogene-addicted tumor cells. While oncogenes indeed drive proliferation, they do so via downstream effector molecules. For example, downstream of ERK signaling is c-MYC, a known regulator of ornithine decarboxylase (ODC) transcription and polyamine biosynthesis (Bello-Fernandez, et al. (1993) Proc. Natl. Acad. Sci., 90(16):7804-7808). The native polyamines (putrescine, spermidine and spermine) are amino acid-derived polycations that have been implicated in a wide array of biological processes, including cellular proliferation, differentiation, chromatin remodeling, eukaryotic initiation factor-5A (eIF-5A) and apoptosis (Casero, et al. (2007) Nat. Rev. Drug Discov., 6(5):373-390). Multiple oncogenes (c-MYC and RAS) are known to up-regulate key polyamine biosynthetic enzymes (Bello-Fernandez, et al. (1993) Proc. Natl. Acad. Sci., 90(16):7804-7808; Forshell, et al. (2010) Cancer Prev. Res., 3(2):140-147; Origanti, et al. (2007) Cancer Res., 67(10):4834-4842) as well as the cellular uptake of polyamines by activating the polyamine transport system (PTS) (Poulin, et al. (2012) Amino Acids 42(2-3):711-723; Bachrach, et al. (1981) Cancer Res., 41(3): 1205-1208; Chang, et al. (1988) Biochem. Biophys. Res. Commun., 157(1):264-270; Roy, et al. (2008) Mol. Carcinog., 47(7):538-553). Compared to normal cells, tumor cells have been shown to contain elevated levels of polyamines (Pegg, A. E. (1988) Cancer Res., 48:759-774; Tabor, et al. (1984) Ann. Rev. Biochem., 53:749-790; Pegg, A. E. (1986) Biochem. J., 234:249-262; Gerner, et al. (2004) Nat. Rev. Cancer 4(10):781-792). These intracellular polyamine levels are maintained via tightly-regulated biosynthetic, catabolic, and uptake and export pathways (Wallace, et al. (2003) Biochem. J., 376(Pt 1): 1-14). Polyamine uptake is upregulated in many tumor types, especially in melanoma tumor cells when compared to normal cells (Poulin, et al. (2012) Amino Acids 42(2-3):711-723; Seiler, et al. (1996) Int. J. Biochem. Cell Biol., 28(8):843-861). Thus, melanoma tumor cells notoriously replete with multiple oncogenic mutations have a greatly increased need for polyamines compared to normal cells to meet their increased metabolic needs (Seiler, et al. (1996) Int. J. Biochem. Cell Biol., 28(8):843-861).

Thus, the oncogene-induced polyamine transport activity in melanoma cells could be exploited by selectively targeting the PTS with a novel arylmethyl-polyamine (AP) compound (Muth, et al. (2013) J. Med. Chem., 56(14):5819-5828). The two-armed design of AP predicated upon a naphthyl core provides PTS hyperselectivity and high potency (Muth, et al. (2013) J. Med. Chem., 56(14):5819-5828). Both exogenous polyamines and polyamine-based drugs are imported into tumors via a specific uptake system (Casero, et al. (2007) Nat. Rev. Drug Discov., 6(5):373-390; Muth, et al. (2013) J. Med. Chem., 56(14):5819-5828; Casero, et al. (2007) Nat. Rev. Drug Discov., 6(5):373-390). Here, it is shown that polyamine uptake is increased in BRAF^{V600E} melanoma cells, and that AP treatment significantly increases cell death in BRAF^{V600E} melanoma cells compared to BRAF^{WT} melanoma cells. Furthermore, it is shown that BRAF inhibitor-resistance in melanoma tumor spheroid cultures can be overcome by treatment with AP. These studies provide valuable insights into developing more effective treatment strategies to restore sensitivity of melanoma tumor cells to BRAF inhibitors. In short, the BRAF-driven polyamine addiction can be targeted by cytotoxic polyamine compounds, which selectively target melanoma cells with high polyamine import activity.

### Materials and Methods

### Cell Lines and Reagents

All human melanoma cell lines including WM983B, WM983B-BR, WM3743, and WM3211, were obtained from the Wistar Institute collection. These cells were maintained in MCDB153 (Sigma)/Leibovitz's L-15 (Cellgro) medium (4:1 ratio) supplemented with 2% fetal calf serum and 2 mmol/L CaCl₂. B16F10 cells were obtained from the ATCC and maintained in DMEM (Invitrogen) supplemented with 10% fetal bovine serum and 100 U/ml penicillin/streptomycin. The YUMM1.7 cell line (Yale University, New Haven, Conn.) was maintained in DMEM/F12 (Invitrogen) medium supplemented with 10% fetal bovine serum and 100 U/ml Penicillin/Streptomycin. PLX4720, (S 1152; Selleckchem, a tool compound related to PLX4032/Vemurafenib) was prepared as a 50 mM stock solution in DMSO and stored at −20° C.

The synthesis of the AP compound was as described (Muth, et al. (2013) J. Med. Chem., 56(14):5819-5828). The compound was dissolved in phosphate buffered saline (PBS) to provide an initial stock (10 mM), which was filtered through a 0.2 m filter to ensure sterility. Subsequent dilutions were made in PBS to generate the desired stock solutions. The structure of AP:

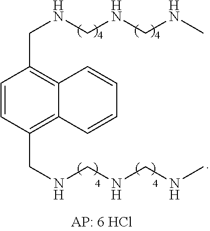

### 3D Spheroid Culture

The inorganic nanoscale scaffolding NanoCulture plates (NCP) were purchased from SCIVAX. The base of each NCP is constructed with a transparent cyclo-olefin resinous sheet with a nanoscale indented pattern. To form spheroids, 1205Lu human melanoma cells were seeded in 96-well NCPs at 1×10⁴ cells/well in MCDB153 (Sigma)/Leibovitz's L-15 (Cellgro) medium (4:1 ratio) supplemented with 2% heat-inactivated fetal calf serum and 2 mM CaCl₂ and incubated in a conventional cell incubator at 37° C. in an atmosphere of 5% CO₂ and normal O₂ levels. When visible spheroids began to form on day 3 after the cells were seeded on the NCPs, treatment with PLX4720 and/or AP was initiated. After drug treatment for 48 hours, the spheroid cultures were assayed for cell viability.

### Cell Viability Assay

Cell proliferation assays were conducted in 96-well plates at 25-30% starting confluence to determine the effect of exposure to increasing concentrations of PLX4720 or AP with or without 1 mM DFMO for 72 hours. Cell viability was assessed using the EZQuant™ Cell Quantifying Kit (Alstem) in which WST-8 is reduced by the metabolic activity of live cells to formazan dye. For spheroids treated with PLX4720 and/or AP, viability of the spheroid cells was estimated by quantification of the ATP present using a CellTiter-Glo Luminescent Cell Viability Assay (Promega Co., Madison, Wis.). The 72 hour IC₅₀ values for AP were calculated using non-linear regression (sigmoidal dose response) of the plot of percentage inhibition versus the log of inhibitor concentration in GraphPad Prism (v5; GraphPad Software, Inc., La Jolla, Calif.).

### Radiolabeled Spermidine Transport Assays

Polyamine transport in tumor cells was evaluated essentially as described (Kramer, et al. (1993) J. Cell Physiol., 155(2):399-407; Nilsson, et al. (2005) Cancer Cell 7(5):433-444). Radioactive spermidine (Net-522 Spermidine Trihydrochloride, [Terminal Methylenes-³H(n)]) from Perkin Elmer was used (specific activity: 16.6 Ci/mmol). Cells were plated in 96 well plates and grown to approximately 80% confluence. Half of the cells were treated with 1 mM DFMO for 40 hours. After repeated washing with PBS, ³H-spermidine was added at 1 μM and incubated for 60 minutes at 37° C. Cells were then washed with cold PBS containing 50 μM spermidine and lysed in 0.1% SDS at 37° C. for 30 minutes with mixing. Cell lysates were then aliquoted for scintillation counting and for protein assay using a mini-BioRad assay. Results were expressed as CPM/g protein.

### Statistical Analysis

All in vitro experiments were performed at least in triplicate, and data were compiled from two to three separate experiments. Analyses were done using a 1-way ANOVA with a Tukey test for statistical significance or a Students t-test. In all cases, values of p≤0.05 were regarded as being statistically significant.

### Results

A panel of human melanoma cell lines with different BRAF mutational status were screened for their sensitivity to the BRAF inhibitor PLX4720. Mutant BRAF^{V600E} melanoma cells, including WM983B, WM3734, 1205Lu, WM989, and WM88, demonstrated marked sensitivity to PLX4720 (IC₅₀ values≤3.0 μM), whereas BRAF^{WT} melanoma cells, including WM3451, WM3743, and WM3211, demonstrated relative resistance to treatment with PLX4720 (IC₅₀>3.0 μM) (Schayowitz, et al. (2012) PloS One, 7(12):e52760). The IC₅₀ value is defined as the concentration of the compound (PLX4720) required to inhibit 50% cell viability compared to an untreated control. This approach allowed for the ranking of the relative sensitivity of each cell line to the PLX4720 BRAF inhibitor. Thus, the sensitivity of these BRAF^{V600E} melanoma cells to BRAF inhibition with PLX4720 reflected their functional dependence on mutant BRAF signaling to sustain their proliferation and viability. Likewise, it was tested whether BRAF^{V600E} cells were more sensitive compared to BRAF^{WT} melanoma cells to increasing concentrations of the cytotoxic polyamine transport ligand, AP.

TABLE 1
Human melanoma cell lines.

| | | AP | | AP + DFMO | |
|---|---|---|---|---|---|
| | | | PTS Activity^{a} | | PTS Activity^{a} |
| | BRAF | IC₅₀ | (cpm ³H | IC₅₀ | (cpm ³H |
| | mutational | (μM | Spd/μg | (μM | Spd/μg |
| Cell Line | status | AP) | Protein) | AP) | Protein) |
| WM983B | V600E | 2.6 | 487 ± 64 | 0.9 | 706 ± 78 |
| WM3734 | V600E | 2.2 | 444 ± 46 | 1.2 | 654 ± 59 |
| 1205Lu | V600E | 0.7 | 230 ± 22 | 0.6 | 299 ± 54 |
| WM989 | V600E | 1.2 | 304 ± 28 | 1.0 | 348 ± 48 |
| WM88 | V600E | 0.8 | 302 ± 18 | 0.2 | 343 ± 59 |
| WM3451 | WT | 9.0 | 130 ± 21 | 5.1 | 157 ± 34 |
| WM3743 | WT | 8.9 | 117 ± 23 | 10.9 | 110 ± 25 |
| WM3211 | WT | 4.5 | 278 ± 58 | 5.2 | 251 ± 46 |

^{a}Polyamine transport system (PTS) activity expressed as CPM ³H Spermidine (Spd)/μg protein ± SD.

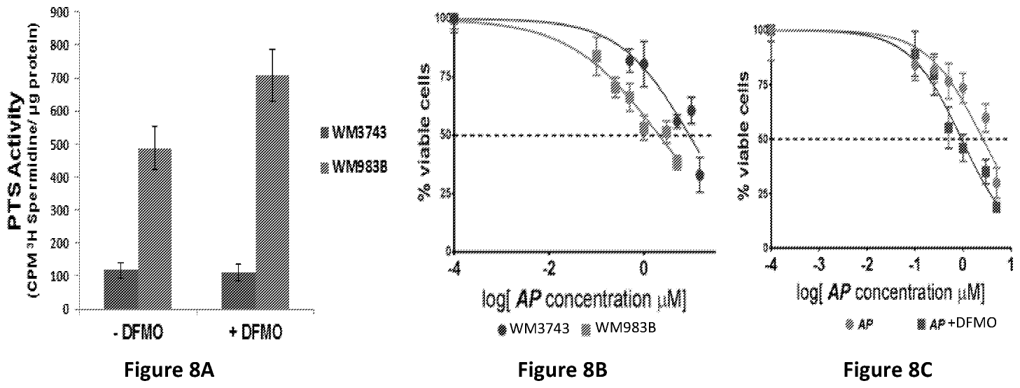

Table 1 shows that BRAF^{V600E} melanoma cells demonstrated greater sensitivity to AP (IC₅₀<2.5 μM) than BRAF^{WT} melanoma cells (IC₅₀>4.0 μM). This observation was reflected by the greater polyamine transport activity in BRAF^{V600E} melanoma cells compared to BRAF^{WT} melanoma cells (FIG. 8A and Table 1). Since AP accumulated at a faster rate in BRAF^{V600E} melanoma cells (such as WM983B) with higher polyamine transport rates compared to BRAF^{WT} melanoma cells (such as WM3743), WM983B cells were more sensitive to AP exposure than WM3743 cells (FIG. 8B). In sum, cell lines with high polyamine import activity were more sensitive to the cytotoxic polyamine compound.

It is well known that lowering intracellular levels of polyamines with inhibitors of polyamine biosynthesis can increase uptake of extracellular polyamines as well as exogenous polyamine analogues (Seiler, et al. (1996) Int. J. Biochem. Cell. Biol., 28(8):843-861; Alhonen-Hongisto, et al. (1980) Biochem. J., 192(3):941-945). WM983B and WM3743 melanoma cells were pretreated for 40 hours with 1 mM α-difluoromethylornithine (DFMO), an inhibitor of ODC, the first and rate-limiting enzyme in polyamine biosynthesis, before measuring their polyamine transport activity. FIG. 8A shows that DFMO treatment dramatically increased polyamine transport activity in BRAF^{V600E} WM983B melanoma cells, but not in the BRAF^{WT}WM3743 melanoma cells. In general, polyamine depletion with DFMO treatment enhanced polyamine uptake more in the screened human BRAF^{V600E} melanoma cells compared to BRAF^{WT} melanoma cells shown in Table 1. Since DFMO treatment increases polyamine transport activity, it was tested whether co-treatment with AP and DFMO will increase the sensitivity of melanoma cells to AP. FIG. 8C shows that WM983B melanoma cells are more sensitive to AP treatment when co-treated with DFMO (IC₅₀=0.7 μM) compared to that with AP alone (IC₅₀=2.3 μM). In contrast, sensitivity to AP was increased in DFMO-co-treated BRAF^{V600E} melanoma cells, but not in BRAF^{WT} melanoma cells (Table 1). These data indicate that human BRAF^{V600E} melanoma cells demonstrate greater polyamine transport activity and increased sensitivity to AP compared to BRAF^{WT} melanoma cells, and their sensitivity can be increased by inhibition of polyamine biosynthesis with DFMO.

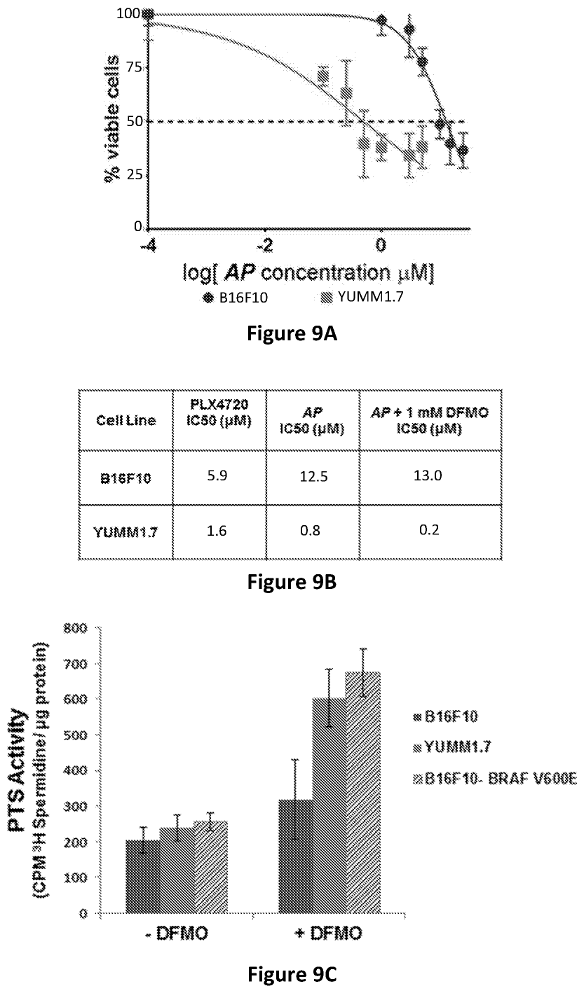

Since human melanoma cells possess multiple oncogenic mutations in addition to BRAF^{V600E}, AP cytotoxicity and PTS activity was compared in the murine B16F10 melanoma cell line that is BRAF^{WT} with BRAF^{V600E} YUMM1.7 cell line that was derived from a melanoma tumor that spontaneously developed in a BRAF^{V600E}/PTEN^{null} transgenic mouse (Obenauf, et al. (2015) Nature 520(7547):368-372). As expected, YUMM1.7 cells were very sensitive to PLX4720 with a lower IC₅₀ compared to B16F10 cells (FIG. 9B). In addition, BRAF^{V600E} YUMM1.7 cells were significantly more sensitive to AP and had a much lower IC₅₀ value for AP compared to BRAF^{WT} B16F10 cells (FIGS. 9A, 9B). In particular, DFMO co-treatment dramatically increased the sensitivity of YUMM1.7 cells to AP, and this correlated with a marked DFMO-induction of PTS activity in BRAF^{V600E} YUMM1.7 cells (FIG. 9C). In contrast, BRAF^{WT} B16F10 cells demonstrated no significant induction in polyamine uptake following DFMO treatment (FIG. 9C). However, B16F10 cells retrovirally infected to express the mutant BRAF^{V600E} protein exhibited a similar PTS activity profile as that seen with YUMM1.7 cells. DFMO treatment was shown to enhance polyamine uptake in the B16F10-BRAF^{V600E} cells as was seen with YUMM1.7 cells (FIG. 9D). AP was also more cytotoxic in B16F10-BRAF^{V600E} cells (IC₅₀=24.4 μM) compared to control-infected B16F10-pBABE cells that were infected with retrovirus expressing the empty plasmid (IC₅₀=36.4 μM). These data indicate that melanoma cells with a mutant BRAF^{V600E} protein are more dependent on the polyamine uptake system compared to cells with a BRAF^{WT} protein, resulting in greater sensitivity to the PTS-targeted cytotoxic AP compound that enters and kills melanoma cells via the polyamine transport system.

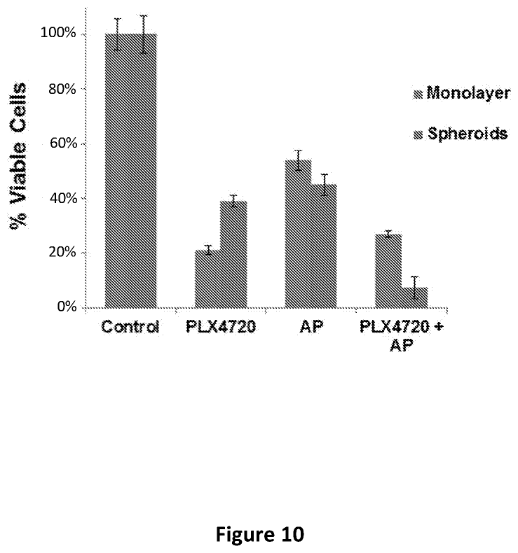

Because growth of cells in a 3D culture system has been found to be more representative of the in vivo microenvironment, 1205Lu human melanoma cells were cultured using an inorganic, nanoscale scaffold-based NanoCulture plate (NCP) in which tumor cells easily form 3D spheroids (Yoshii, et al. (2011) Biomaterials 32(26):6052-6058). Although these tumor spheroid cultures are grown in ambient air, the spheroid microenvironment closely resembles that in tumors with a hypoxic core and is more relevant for drug sensitivity compared to that seen with monolayer cultures (Yoshii, et al. (2011) Biomaterials 32(26):6052-6058; Yamada, et al. (2007) Cell 130(4):601-610; Haycock, J. W., 3D cell culture: a review of current approaches and techniques. 3D cell culture: methods and protocols, 1-15). BRAF^{V600E} mutant 1205Lu melanoma cells grown as spheroids on NCPs were more resistant to 48 hours treatment with PLX4720 (25 μM) compared to the same cells grown in 2D monolayer cultures in ambient air (FIG. 10) (Qin, et al. (2016) Mol. Cancer Therap., 15(10): 2442-2454). Both spheroid and monolayer cultures were similarly sensitive to 48 hour treatment with a high concentration of AP (25 μM) alone. The effect of AP treatment on the PLX4720-resistant phenotype of the 1205Lu spheroid and monolayer cultures was tested. Co-treatment with both PLX4720 (25 μM) and AP (25 μM) led to a dramatic reduction in cell viability in the spheroid cultures unlike monolayer cultures that showed no further reduction in cell viability when compared to PLX4720 treatment alone (FIG. 10). Thus, the increased resistance of the melanoma spheroid cultures to PLX4720 was eliminated with AP co-treatment.

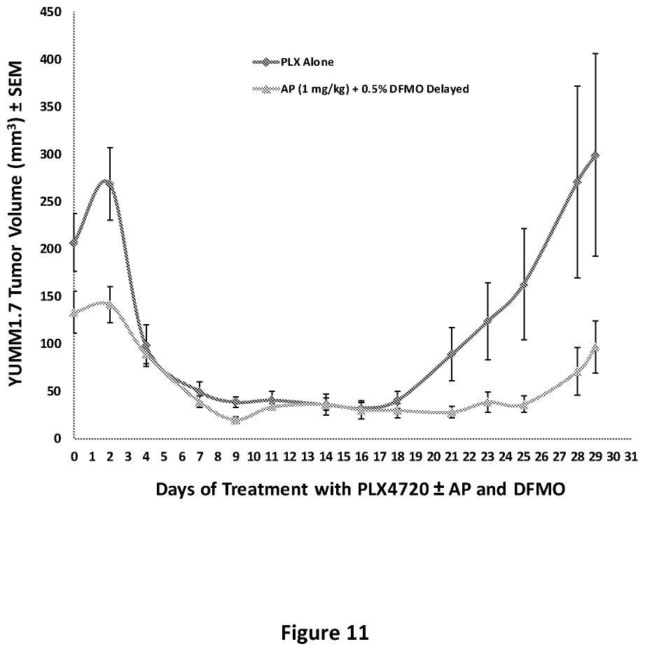

In addition to the above, the efficacy of AP in vivo was also tested. As seen in FIG. 11, the administration of the BRAF inhibitor PLX4720 causes a BRAF-mutant melanoma tumor in mice to initially quickly regress but then grow back. However, co-treatment with AP, beginning when the tumor has regressed, significantly retards the regrowth of a BRAF inhibitor-resistant tumor (FIG. 11). These data indicate that utilizing the PTS for drug delivery with AP attacks mutant BRAF melanoma tumor cells via one of their key modes of survival.

The data provided herein show that melanoma tumor cells expressing mutant BRAF^{V600E} exhibit a high demand for polyamine growth factors and a greatly upregulated polyamine transport system (PTS). Utilizing the PTS for drug delivery, the AP compound attacks the melanoma cells via one of its key modes of survival. Indeed, polyamines are essential for the survival of melanomas. Polyamine levels are dramatically elevated in tumor cells compared to normal cells, often the result of oncogenic induction (Poulin, et al. (2012) Amino Acids 42(2-3):711-723; Seiler, et al. (1996) Int. J. Biochem. Cell. Biol., 28(8):843-861). The MYC and RAS oncogenes can upregulate polyamine biosynthesis (Forshell, et al. (2010) Cancer Prev. Res., 3(2):140-147; Origanti, et al. (2007) Cancer Res., 67(10):4834-4842) and increase cellular uptake of polyamines by inducing PTS activity (Bachrach, et al. (1981) Cancer Res., 41(3):1205-1208; Chang, et al. (1988) Biochem. Biophys. Res. Commun., 157(1):264-270; Roy, et al. (2008) Mol. Carcinog., 47(7):538-553). Although melanoma cells are notoriously replete with multiple oncogenic mutations, more than half of all melanoma tumors express a mutant BRAF protein (Seiler, et al. (1996) Int. J. Biochem. Cell. Biol., 28(8):843-861). The data herein indicate that BRAF^{V600E} melanoma tumors have a greatly increased metabolic need for polyamines compared to normal cells. The BRAF^{V600E}-induced PTS activity in metastatic melanoma cells was exploited by targeting the PTS with AP.

Putrescine, spermidine, and spermine play key roles in cellular proliferation, signal transduction, gene expression, and autophagic states that contribute to tumor survival (Cufi, et al. (2011) Cell Cycle 10(22):3871-3885; Mirzoeva, et al. (2011) J. Mol. Med., 89(9):877-889; Morselli, et al. (2011) Antioxid. Redox Signal 14(11):2251-2269; Morselli, et al. (2011) J. Cell. Biol., 192(4):615-629). These endogenous polyamines and the polyamine-based AP compete to be imported into tumors via the PTS (Muth, et al. (2013) J. Med. Chem., 56(14):5819-5828). However, arylmethyl-polyamine drugs like AP may have enhanced cytotoxic potency via their multiple electrostatic interactions with DNA (Dallavalle, et al. (2006) J. Med. Chem., 49(17):5177-5186) and topoisomerase II (Wang, et al. (2001) Mol. Cell. Biochem., 227(1-2):167-74). Since AP selectively targets tumor cells with high polyamine transport rates, normal cells are significantly less sensitive to AP since they have low PTS activity (Muth, et al. (2013) J. Med. Chem., 56(14):5819-5828). Polyamine biosynthesis and cellular uptake are induced in hypoxic regions of tumors and in tumor spheroids (Svensson, et al. (2008) Cancer Res., 68(22):9291-9301). Moreover, depletion of polyamines during hypoxia resulted in increased apoptosis (Svensson, et al. (2008) Cancer Res., 68(22):9291-9301), indicating that polyamines play an essential role in the ability of tumor cells to adapt to hypoxic stress and reactive oxygen species. Indeed, polyamines are known to exert anti-oxidant functions (Mozdzan, et al. (2006) Int. J. Biochem. Cell Biol., 38(1):69-81). Although the melanoma spheroid cultures in this study were cultured with ambient air, it is well documented that the cells at the center of spheroids are hypoxic, thus modeling the heterogeneous 3D structure of in vivo melanoma tumors that often contain hypoxic regions (Yoshii, et al. (2011) Biomaterials 32(26):6052-6058; Qin, et al. (2016) Mol. Cancer Ther., 15(10):2442-2454). Solid tumors contain poorly vascularized, hypoxic regions that contribute to tumor progression by activating a hypoxia stress response via hypoxia inducible factor-1α (HIF-1α) that promotes cell survival, tumor angiogenesis, and metastasis (Pouyssegur, et al. (2006) Nature 441(7092):437-443; Keith, et al. (2007) Cell 129(3):465-472). Hypoxic tumor cells and spheroid cultures are more resistant to chemotherapy including BRAF inhibitors (Qin, et al. (2016) Mol. Cancer Ther., 15(10):2442-2454; O'Connell, et al. (2013) Cancer Discov., 3(12):1378-1393; Pucciarelli, et al. (2016) Mol. Med. Rpts., 13(4):3281-3288). Likewise, 3D cultures of 1205Lu melanoma cells grown as spheroids on NCPs are more resistant to PLX4720 treatment compared to 1205Lu cells grown in 2D monolayer culture in ambient air. Knowing that polyamine uptake is induced in hypoxic regions of tumor spheroids, treatment with the PTS ligand AP increases the sensitivity of 1205Lu spheroid cells to PLX4720. Indeed, the increased resistance of melanoma spheroids to PLX4720 was overcome with AP co-treatment.

Treatment with a BRAF inhibitor such as PLX4720 enriches a slow-cycling cancer stem cell-like (CSC) subpopulation of melanoma cells that is characterized by stem cell markers including JARID1B and spheroid formation (Roesch, et al. (2010) Cell 141(4):583-594; Roesch, et al. (2013) Cancer Cell 23(6):811-825). It is thought that cancer stem cell populations exist in a hypoxic microenvironment (Schwab, et al. (2012) Breast Cancer Res., 14(1):R6; Mathieu, et al. (2011) Cancer Res., 71(13):4640-4652; Mohyeldin, et al. (2010) Cell Stem Cell, 7(2):150-161). Endogenous ROS levels are increased in slow cycling JARID1B^{high} melanoma cells as a result of increased mitochondrial respiration and oxidative phosphorylation (Roesch, et al. (2013) Cancer Cell 23(6):811-825). This high oxygen consumption contributes to hypoxic conditions that have been shown to favor the JARID1B^{high} slow-cycling CSC-like phenotype (Roesch, et al. (2010) Cell 141(4):583-594). Using 1205Lu melanoma cells stably transduced with a JARID1B-promoter-EGFP-reporter construct (Roesch, et al. (2010) Cell 141(4):583-594), it was found that a short 2-day exposure to PLX4720 led to a 4-fold enrichment of JARID1B-driven GFP expressing 1205Lu melanoma cells grown as spheroids. The data provided herein show that PLX4720-resistant melanoma spheroids are made more sensitive to PLX4720 with AP co-treatment, and that AP is likely targeting CSC subpopulations that are enriched in the PLX4720-resistant melanoma spheroids.

Polyamines may also contribute to tumor survival by inducing an autophagic state (Cufi, et al. (2011) Cell Cycle 10(22):3871-3885; Mirzoeva, et al. (2011) J. Mol. Med., 89(9):877-889; Morselli, et al. (2011) Antioxid. Redox Signal 14(11):2251-2269; Morselli, et al. (2011) J. Cell. Biol., 192(4):615-629). For instance, spermidine has been shown to induce autophagy in multiple systems including yeast cells, C. elegans, Drosophila, and human tumor cells (Morselli, et al. (2011) J. Cell. Biol., 192(4):615-629; Eisenberg, et al. (2009) Nat. Cell. Biol., 11(11):1305-1314) and to increase survival of pluripotent stem cells in culture (Chen, et al. (2011) Aging Cell 10(5):908-911). Autophagy has recently emerged as a common survival process that tumors undergo when assaulted by chemotherapy and radiation (Strohecker, et al. (2014) Cancer Discov., 4(7):766-772). It is induced by cellular stress such as nutrient deprivation, withdrawal of growth factors, and hypoxia (Kroemer, et al. (2010) Mol. Cell., 40(2):280-293). In established tumors, autophagy is also a resistance mechanism to many therapeutic modalities including BRAF inhibitors (Ma, et al. (2014) J. Clin. Invest., 124(3):1406-1417). Increased polyamine uptake provides a mechanism for BRAFi-resistant melanoma cells to acquire sufficient polyamines to undergo autophagy to survive treatment with BRAF inhibitors.

Clinical trials have tested the anti-tumor efficacy of the ODC inhibitor DFMO. However, treatment with DFMO alone demonstrated only moderate success in treating cancer patients (Seiler, N. (2003) Curr. Drug Targets 4(7):537-564). Subsequent studies discovered that DFMO-inhibition of polyamine biosynthesis leads to upregulation of PTS activity with resulting increased uptake of polyamines from the diet and gut flora into the tumor cells (Wallace, et al. (2003) Biochem. J., 376(Pt 1): 1-14). The findings provided herein show that DFMO induces PTS activity and increases AP sensitivity in melanoma cells that harbor a mutated BRAF protein. In summary, treatment with AP, with or without DFMO, offers exciting potential as adjunct cancer therapy to overcome drug resistance in BRAF^{V600E} melanoma.

### Example 3

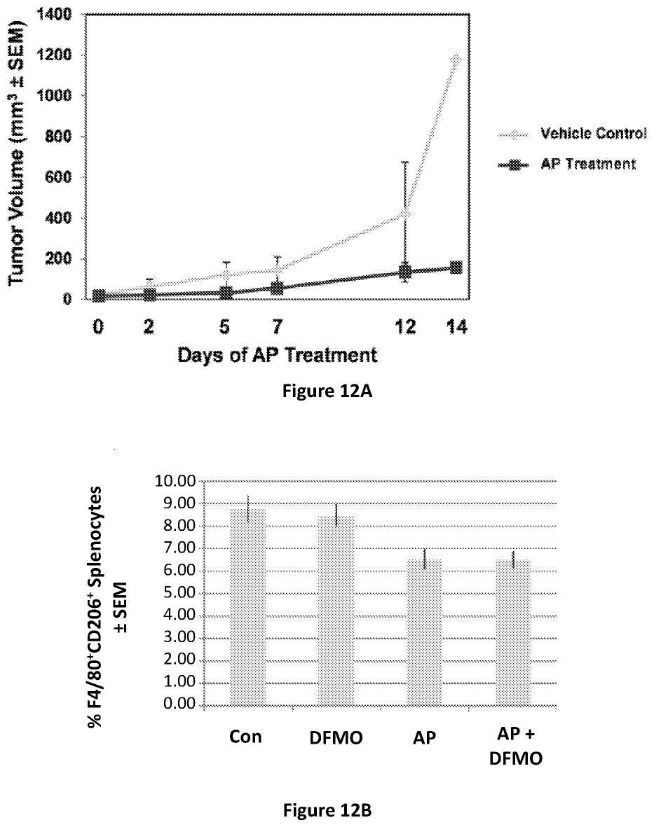

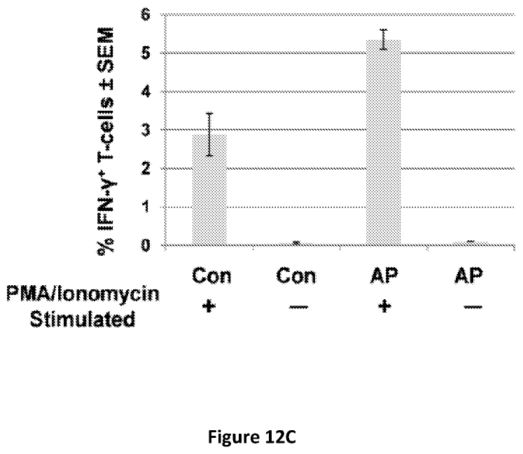

C57Bl/6 mice were injected s.c. with 1.5×10⁵ B16F10 melanoma cells. B16F10 melanoma cells are a poorly immunogenic, highly metastatic, and highly aggressive cancer cell line. When tumors were 40-80 mm³ in size, AP (Formula (II)) treatment (i.p., 2 mg/kg every other day) was initiated. Some mice were also administered 0.25% DFMO (w/v) in the drinking water. Tumors were measured with calipers to determine tumor volume over 14 days of treatment (FIG. 12A). Splenocytes from mice treated with vehicle, DFMO, AP, or AP+DFMO were analyzed by flow cytometry for F4/80+CD206⁺ M2 macrophages (FIG. 12B). Splenocytes were treated for 4 hours at 37° C. with ionomycin (500 ng/ml) and PMA (50 ng/ml) to stimulate T cells to produce IFNγ in the presence of Brefeldin A to block cytokine secretion. Cells were stained with anti-CD4 antibody in the presence of Brefeldin A, fixed, permeabilized and intracellularly stained with an anti-IFNγ antibody and analyzed by flow cytometry (FIG. 12C). As seen in FIG. 12, treatment with AP alone inhibits tumor growth in mice and dramatically decreases CD206⁺ F4/80⁺ macrophages (immunosuppressive) while increasing IFN-gamma-secreting T cells in tumor-bearing mice.

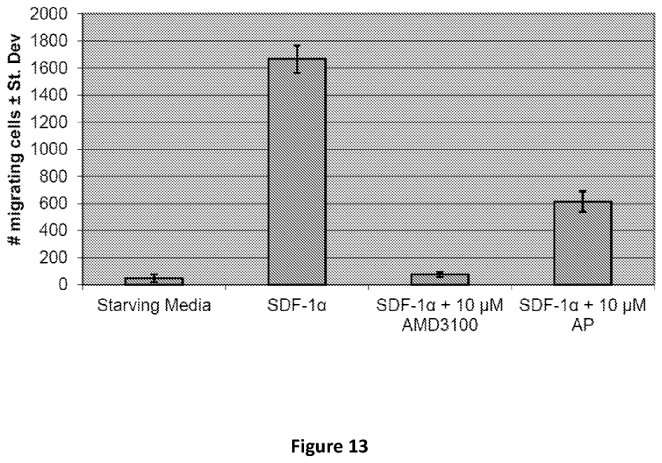

The ability to inhibit stromal cell-derived factor 1 (SDF-1) stimulated migration of macrophage was also tested in an in vitro chemotaxis assay. Specifically, CXCR4⁺ RAW247.6 macrophage cells were treated with SDF-1α alone or with AMD3100, a specific CXCR4 antagonist which inhibits cell migration and phosphorylation of ERK1/2 by SDF-1, or AP (Formula (II)). As seen in FIG. 13, AP inhibits SDF-1-stimulated migration of CXCR4⁺ macrophages in an in vitro chemotaxis assay without affecting the viability of the macrophages.

Several publications and patent documents are cited in the foregoing specification in order to more fully describe the state of the art to which this invention pertains. The disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1: A method for treating, inhibiting, and/or preventing a cancer in a subject in need thereof, said method comprising administering:
A) a polyamine transport system inhibitor to said subject, wherein said polyamine transport system inhibitor has the structure: 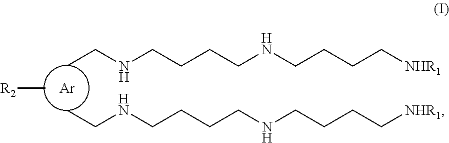
wherein each R₁ is independently H or methyl, wherein Ar is an aryl group, and wherein R₂ is H or 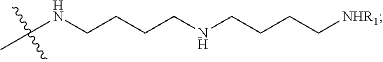
and
B) an anti-cancer therapy to said subject.

2: A method for increasing the therapeutic efficacy of an anti-cancer therapy, said method comprising administering a polyamine transport system inhibitor with said anti-cancer therapy, wherein said polyamine transport system inhibitor has the structure: 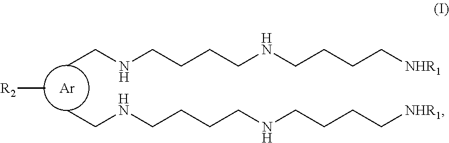
wherein each R₁ is independently H or methyl, wherein Ar is an aryl group, and wherein R₂ is H or

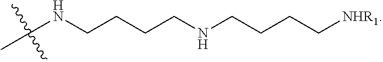

3: The method of claim 1, wherein Ar is selected from the group consisting of benzene, naphthalene, and anthracene.

4: The method of claim 1, further comprising administering an ornithine decarboxylase inhibitor.

5: The method of claim 4, wherein said ornithine decarboxylase inhibitor is a-difluoromethyl-ornithine (DFMO).

6: The method of claim 1, wherein said cancer is metastatic.

7: The method of claim 1, wherein said cancer is a melanoma.

8: The method of claim 1, wherein said cancer comprises CXCR4 positive cells.

9: The method of claim 1, wherein said cancer is resistant to said anti-cancer therapy.

10: The method of claim 1, wherein said anti-cancer therapy is chemotherapy, radiation therapy, surgery, and/or immunotherapy.

11: The method of claim 1, wherein said polyamine transport system inhibitor has the structure: 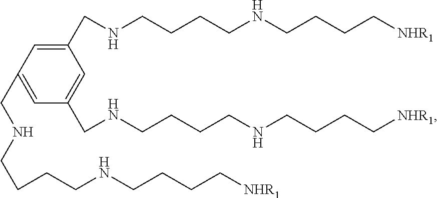
wherein each R₁ is independently H or methyl.

12: A method for modulating the immune system of a subject, said method comprising administering a polyamine transport system inhibitor to said subject, wherein said polyamine transport system inhibitor has the structure: 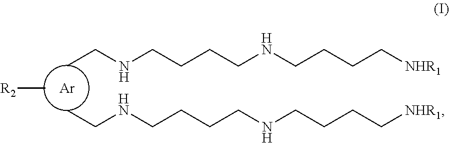
wherein each R₁ is independently H or methyl, wherein Ar is an aryl group, and wherein R₂ is H or

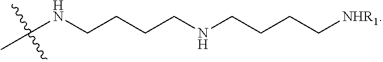

13: The method of claim 12, wherein Ar is selected from the group consisting of benzene, naphthalene, and anthracene.

14: The method of claim 12, further comprising administering an ornithine decarboxylase inhibitor.

15: The method of claim 14, wherein said ornithine decarboxylase inhibitor is a-difluoromethyl-ornithine (DFMO).

16: The method of claim 12, wherein said polyamine transport system inhibitor has the structure: 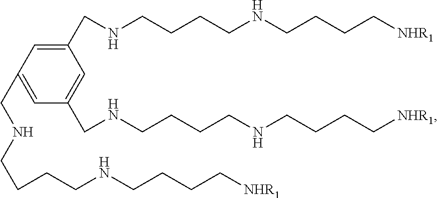
wherein each R₁ is independently H or methyl.

17: The method of claim 12, wherein said method increases proinflammatory cytokines in said subject.

18: The method of claim 17, wherein said proinflammatory cytokines include at least one of IL-10, IFN-γ, and MCP-1.

19: The method of claim 12, wherein said method increases the immune response in said subject.

20: The method of claim 2, wherein Ar is selected from the group consisting of benzene, naphthalene, and anthracene.

21: The method of claim 2, further comprising administering an ornithine decarboxylase inhibitor.

22: The method of claim 2, wherein said cancer is metastatic.

23: The method of claim 2, wherein said cancer is a melanoma.

24: The method of claim 2, wherein said cancer comprises CXCR4 positive cells.

25: The method of claim 2, wherein said cancer is resistant to said anti-cancer therapy.

26: The method of claim 2, wherein said anti-cancer therapy is chemotherapy, radiation therapy, surgery, and/or immunotherapy.

27: The method of claim 2, wherein said polyamine transport system inhibitor has the structure: 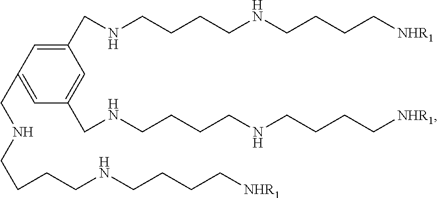
wherein each R₁ is independently H or methyl.


SEQUENCE LISTING (2 sequences)

SEQ ID NO: 1 - protein, 8 aa, Artificial Sequence
  Synthetic Peptide
SIINFEKL

SEQ ID NO: 2 - protein, 9 aa, Artificial Sequence
  Beta-galactosidase Peptide
TPHPARIGL